SEQUENCE LISTING (44 sequences)

SEQ ID NO: 1 - protein, 43 aa, Homo sapiens
DAEFRHDSGYEVHHQKLVFFAEDVGSNKGAIIGLMVGGVVIAT

SEQ ID NO: 2 - protein, 15 aa, Homo sapiens
DAEFRHDSGYEVHHQ

SEQ ID NO: 3 - protein, 15 aa, Clostridium tetani
  MISC_FEATURE (15)..(15): Xaa can be absent or Leu
QYIKANSKFIGITEX

SEQ ID NO: 4 - protein, 21 aa, Clostridium tetani
FNNFTVSFWLRVPKVSASHLE

SEQ ID NO: 5 - protein, 20 aa, Clostridium tetani
IDKISDVSTIVPYIGPALNI

SEQ ID NO: 6 - protein, 20 aa, Clostridium tetani
NSVDDALINSTKIYSYFPSV

SEQ ID NO: 7 - protein, 17 aa, Clostridium tetani
PGINGKAIHLVNNESSE

SEQ ID NO: 8 - protein, 14 aa, Clostridium tetani
ZYIKANSKFIGITE

SEQ ID NO: 9 - protein, 21 aa, Plasmodium falciparum
DIEKKIAKMEKASSVFNVVNS

SEQ ID NO: 10 - protein, 16 aa, Streptococcus mutans
GAVDSILGGVATYGAA

SEQ ID NO: 11 - protein, 13 aa, Artificial Sequence
  chemically synthesized
XKXVAAWTLKAAX

SEQ ID NO: 12 - protein, 15 aa, Clostridium tetani
QYIKANSKFIGITEL

SEQ ID NO: 13 - protein, 34 aa, Artificial Sequence
  chemically synthesized
CSSQYIKANSKFIGITELSDAEFRHDSGYEVHHQ

SEQ ID NO: 14 - protein, 28 aa, Artificial Sequence
  chemically synthesized
CSSQYIKANSKFIGITELDAEFRHDSGY

SEQ ID NO: 15 - protein, 32 aa, Artificial Sequence
  chemically synthesized
QYIKANSKFIGITELSKDAEFRHDSGYEVHHQ

SEQ ID NO: 16 - protein, 32 aa, Artificial Sequence
  chemically synthesized
QYIKANSKFIGITELSDAEFRHDSGYEVHHQK

SEQ ID NO: 17 - protein, 32 aa, Artificial Sequence
  chemically synthesized
DAEFRHDSGYEVHHQKSQYIKANSKFIGITEL

SEQ ID NO: 18 - protein, 34 aa, Artificial Sequence
  chemically synthesized
CSSKDAEFRHDSGYEVHHQQYIKANSKFIGITEL

SEQ ID NO: 19 - protein, 34 aa, Artificial Sequence; identical to SEQ ID NO: 13
  chemically synthesized

SEQ ID NO: 20 - protein, 34 aa, Artificial Sequence; identical to SEQ ID NO: 13
  chemically synthesized

SEQ ID NO: 21 - protein, 31 aa, Artificial Sequence
  chemically synthesized
CSSXKXVAAWTLKAAXDAEFRHDSGYEVHHQ

SEQ ID NO: 22 - protein, 31 aa, Artificial Sequence; identical to SEQ ID NO: 21
  chemically synthesized

SEQ ID NO: 23 - protein, 31 aa, Artificial Sequence
  chemically synthesized
QYIKANSKFIGITELSDAEFRHDSGYEVHHQ

SEQ ID NO: 24 - protein, 28 aa, Artificial Sequence
  chemically synthesized
XKXVAAWTLKAAXDAEFRHDSGYEVHHQ

SEQ ID NO: 25 - protein, 16 aa, Artificial Sequence
  chemically synthesized
SDAEFRHDSGYEVHHQ

SEQ ID NO: 26 - protein, 20 aa, Artificial Sequence
  chemically synthesized
CSSKSDAEFRHDSGYEVHHQ

SEQ ID NO: 27 - protein, 21 aa, Artificial Sequence
  chemically synthesized
CSSKDAEFRHDSGYEVHHQST

SEQ ID NO: 28 - protein, 13 aa, Artificial Sequence
  chemically synthesized
XKXVAAWTLKAAX

SEQ ID NO: 29 - protein, 34 aa, Artificial Sequence; identical to SEQ ID NO: 13
  chemically synthesized

SEQ ID NO: 30 - protein, 37 aa, Artificial Sequence
  chemically synthesized
CSKKKKSQYIKANSKFIGITELDAEFRHDSGYEVHHQ

SEQ ID NO: 31 - protein, 41 aa, Artificial Sequence
  chemically synthesized
CSSQYIKANSKFIGITELSKKKKKKSDAEFRHDSGYEVHHQ

SEQ ID NO: 32 - protein, 43 aa, Artificial Sequence
  chemically synthesized
CSKKKKSQYIKANSKFIGITELSKKKKSDAEFRHDSGYEVHHQ

SEQ ID NO: 33 - protein, 47 aa, Artificial Sequence
  chemically synthesized
CSKKKKKKSQYIKANSKFIGITELDAEFRHDSGYEVHHQSKKKKKKS

SEQ ID NO: 34 - protein, 32 aa, Artificial Sequence
  chemically synthesized
CSKKKKSQYIKANSKFIGITELDAEFRHDSGY

SEQ ID NO: 35 - protein, 4 aa, Artificial Sequence
  linker sequence
KKKK

SEQ ID NO: 36 - protein, 6 aa, Artificial Sequence
  linker sequence
KKKKKK

SEQ ID NO: 37 - protein, 4 aa, Artificial Sequence
  linker sequence
KEKE

SEQ ID NO: 38 - protein, 4 aa, Artificial Sequence
  linker sequence
EEEE

SEQ ID NO: 39 - protein, 6 aa, Artificial Sequence
  linker sequence
SKKKKS

SEQ ID NO: 40 - protein, 8 aa, Artificial Sequence
  linker sequence
SKKKKKKS

SEQ ID NO: 41 - protein, 6 aa, Artificial Sequence
  linker sequence
GKKKKG

SEQ ID NO: 42 - protein, 8 aa, Artificial Sequence
  linker sequence
GKKKKKKG

SEQ ID NO: 43 - protein, 6 aa, Artificial Sequence
  linker sequence
SKEKES

SEQ ID NO: 44 - protein, 7 aa, Artificial Sequence
  chemically synthesized
CSKKKKS

(19) United States
(12) Patent Application Publication
(10) Pub. No.: US 2009/0123488 A1
(43) Pub. Date: May 14, 2009

(54) Compositions and methods for the treatment and prophylaxis of Alzheimer's disease

(75) Inventor: Gideon Goldstein, Short Hills, NJ (US)

Correspondence Address: HOWSON & HOWSON LLP, 501 OFFICE CENTER DRIVE, SUITE 210, FORT WASHINGTON, PA, 19034, US

(73) Assignee: Thymon, LLC, Short Hills, NJ (US)

(21) Appl. No.: 11/891,880

(22) Filed: Aug. 14, 2007

Related U.S. Application Data
(60) Provisional application No. 60/837,521, filed on Aug. 14, 2006.

(51) Int. Cl. A61K 39/385; A61P 37/04

(52) U.S. Cl. 424/185.1

(57) ABSTRACT

A self-adjuvanting immunogenic composition comprising an immunogen comprising a lipopeptide cap (R2), a universal T helper sequence (R1) and an immunodominant Aβ B cell epitope. The immunogen also comprises one or more linker sequences and/or polar charged amino acid sequences. The B cell epitope of each immunogen has an amino acid sequence located within the first 17 amino acids of SEQ ID NO: 1. The lipopeptide is a dipalmitoyl-S-glyceryl-cysteine or a tripalmitoyl-S-glyceryl cysteine or N-acetyl (dipalmitoyl-S-glyceryl cysteine), each with an optional neutral amino acid linker. Optional polar sequences of at least four charged polar amino acids enhance solubility of the immunogen and are located at the carboxy terminal end of R2, optionally flanked by neutral linker amino acids, or elsewhere in the immunogen. Such compositions, at surprisingly low dosages of less than 10 mg per subject, can induce anti-Aβ peptide antibodies with GMTs of 50,000 or greater than 1,000,000 when employed to immunize a mammalian subject, without any extrinsic adjuvant.

### CROSS-REFERENCE TO RELATED APPLICATIONS

This application claims the benefit under 35 USC 119(e) of prior U.S. Provisional Patent Application No. 60/837,521, filed Aug. 14, 2006.

### BACKGROUND OF THE INVENTION

Alzheimer's disease (AD) is a progressive dementia that is associated with abnormal accumulation of a peptide referred to as amyloid-β (Aβ) or β-amyloid into extracellular toxic plaques (Schenk, D., 2002 Nat Rev Neuroscience 3:825). These plaques have been considered to be responsible for neurodegeneration and resulting dementia, an hypothesis that was supported by a growing body of experimental and clinical evidence. However, more recent evidence suggests that soluble amyloid β is directly neurotoxic, and that accumulation of intracellular amyloid β within neurons is implicated in neurotoxicity (Oddo et al, 2006 J. Biol. Chem., 51:39413; Oddo et al, 2006, Am. J. Pathol., 168:184; Tong et al, 2004 J. Neurosci., 24:6799).

The amyloid-β peptide (Aβ) is an internal fragment of 39-43 amino acids, which is cleaved from the amyloid precursor protein (APP), a protein that has isoforms of 695, 751 and 770 amino acids in length, by β- and γ-secretases. See, e.g., U.S. Pat. No. 4,666,829; Glenner & Wong, 1984 Biochem., Biophys Res. Commun. 120:1131. The amino acid residue 598 of APP is the first amino acid of the amyloid β peptide. The 43 amino acid sequence of amyloid β peptide is shown as SEQ ID NO: 1. Aβ15 or Aβ10 refers to a peptide containing amino acid residues 1-15 or 1-10 of amyloid β and Aβ40 refers to a peptide containing amino acid residues 1-40 of amyloid β, etc. See, United See Patent Application Publication No. US2004/0213800, incorporated by reference herein.

Research involving immunizations with Aβ peptides in mouse models of AD (Schenk et al. 1999 Nature 400:173; Janus et al. 2000 Nature 408:979; Morgan et al. 2000 Nature 408:982; Sigurdsson et al. 2001 Am J Pathol 159:439; McLaurin et al. 2002 Nat Med 8:1263; and Levites et al. 2006 J Clin Invest 116:193) provides a remarkably consistent picture. Immunization with Aβ40 (e.g., Aβ1-40, or the closely related aa1-42 or aa1-43 forms), slowed the appearance of and/or ameliorated established neuropathology, and functional neurological parameters, as measured by an array of tests, in several animal models of AD. Immunizations were performed using complete Freund's adjuvant (CFA) for priming and incomplete Freund's adjuvant (IFA) for boosting at 2 weeks and monthly subsequently. Anti-amyloid antibody titers ranged from a few hundred to several thousand (Sigurdsson et al., 2001 cited above) to 5,000 to 50,000 with CFA (McLaurin et al., cited above). Similar immunizations have been reported with Aβ28 (Solomon et al 1997 Proc Natl Acad Sci USA 94:4109) and Aβ33 (Agadjanyan et al 2005 J Immunol 174:1580). US Patent Application Publication No. US2004/0213800 also deals with active immunization to generate antibodies to soluble Aβ peptide by administering Aβ fragments from the C terminal of the peptide. Such peptides include Aβ15 or Aβ10-24 and subfragments of 5-10 contiguous amino acids thereof. Antibody titers from an ELISA using mouse serum showed titers of between about 3600 to 14,457.

Passive immunotherapy using monoclonal antibodies to Aβ peptides also confirmed that immunotherapeutic reduction of circulating Aβ peptide by antibody could prevent the development of neuropathology and neurological deterioration in animals models, and also ameliorate these parameters in early established disease (Bard et al. 2000 Nat Med 6:916; Dodart et al. 2002 Nature 5:452).

A trial of amyloid-β immunization in mild to moderate AD patients was reported in Hock et al. 2003 Neuron 38:547. Immunization was with amyloid-β42 and the experimental saponin adjuvant QS-21, given at baseline and at months 1, 3, 6, 9 and 12. The study was terminated after several patients developed transient episodes of immunization-associated aseptic meningoencephalitis (ME). Despite this, analysis of the clinical parameters showed striking and statistically significant slowing of cognitive decline in the subset of patients that developed anti-amyloid-β titers of 2,200 to 4,000 (20 subjects) in contrast to continued decline in those with antibody titers less than 2,200. More extensive follow-up of these patients was reported by Orgogozo et al. 2003 Neurology 61:46 for the ME, and by Gilman et al 2005 Neurology 64:1553 for the clinical response follow-up. Evidence for ME occurred in 18/298 (6%) of treated patients versus 0/74 controls. There was no correlation with antibody titers and the authors speculated that T cell immunity may have triggered an autoimmune reaction.

Despite the trial interruption and incomplete treatments in many patients the composite score across the neuropsychological test battery was statistically significant (P=0.02) for antibody responders versus non-responders. However, only 59/300 (20%) of treated patients developed antibody titers ≧2,200 and the maximal titers were 4,000. In summary, the trial provided proof of principle but was hampered by poor immunogenicity, likely due to inability to use strong adjuvants such as CFA/IFA in humans versus their use in animal experiments. The unacceptable occurrence of ME, likely due to a damaging T cell mediated autoimmune response, provides a further obstacle to development of this approach.

The potential utility of antibodies directed to epitopes within shorter N-terminal peptides of amyloid-β was examined in animal studies (Solomon et al 1997 Proc Natl Acad Sci USA 94:4109; McLaurin et al 2002 Nat Med 8:1263; and Bard et al 2003 Proc Natl Acad Sci USA 100:2023). However, the shorter peptides are reported to be even less immunogenic and require CFA/IFA adjuvant to get adequate titers.

Both the problems of immunogenicity and the T cell response issue were addressed by Agadjanyan et al 2005 J Immunol 174:1580, who synthesized Aβ33 and Aβ115 with or without the sequence of a promiscuous foreign T cell epitope, termed PADRE, either as a linear sequences or as a multiple antigenic peptides (Tam, 1988 Proc. Natl. Acad. Sci. USA, 85:5409-5413). Alum, a mild adjuvant suitable for human use, was used. With five boosts the maximal titer was 64,000 for Aβ33-MAP and 32,000 for Aβ33 and PADRE-Aβ15-MAP. Importantly, Agadjanyan et al demonstrated that Aβ33-MAP immunized mice had a demonstrable T cell response to Aβ40, whereas PADRE-Aβ15-MAP immunized mice did not.

Maier et al, 2006 J. Neurosci, 26:4717 also demonstrated that immunization with amyloid β 1-15 reduced cerebral amyloid β load and learning deficits in an Alzheimer's Disease mouse model in the absence of amyloid β-specific cellular immune responses. Additional studies by Wong et al, 2007 Vacc., 25:3041 similarly showed that amyloid β 1-14 in a proprietary adjuvant formulation induced an anti-amyloid β antibody response without evoking anti-amyloid β cellular responses in a transgenic mouse model of Alzheimer's disease.

Despite the foregoing studies and the plethora of patent literature in the field of the treatment of Alzheimer's disease, there remains a need in the art for new and useful compositions and methods for generating a therapeutic/prophylactic immunogenic composition for Alzheimer's disease, which does not result in adverse side effects. There remains a need for potent Aβ immunogens capable of inducing high and persistent antibody titers to amyloid without the addition of potent adjuvants, if a successful composition for the treatment and/or prophylaxis of Alzheimer's disease in humans is to be attained.

### BRIEF DESCRIPTION OF THE DRAWINGS

FIG. 1 is a graph showing the geometric mean titers (GMT) induced by Aβ15 (also referred to as Aβ1-15) immunogens with no added adjuvant. These results were developed with serums from immunized mice. The immunogens identified under the X axis of FIG. 1 included experimental immunogens (Pam2-QYIK-AB1-15, Pam3-QYIK-AB1-15, Pam2-Padre-AB1-15 and Pam3-Padre-AB1-15) and less effective immunogens (QYIK-AB1-15 and Padre-AB1-15) synthesized as described in Example 1 and identified in that example. The serum of mice immunized with the experimental immunogens showed GMTs greater than 50,000, which were dramatically higher than the GMTs of the control immunogens, which were less than about 5000. These results demonstrate the need for a lipopeptide cap and a universal T helper sequence in the experimental immunogens to obtain humoral enhancement of the immune response.

FIG. 2 is a graph showing antibody titer vs. percent inhibition as measured against Aβ40 by mouse anti-Aβ15 antiserum. This dose response curve of inhibition of free Aβ40 concentration in relation to antibody titer shows 50% inhibition (reduction) of free Aβ40 concentration at a titer of 28,000, a titer below the GMT obtained with the 1 mg prime/boost regimen (no adjuvant) used with the effective immunogens. Extrapolation of the curve suggests that a titer of 120,000 would provide 90% reduction of free Aβ concentration.

FIG. 3A is a chemical structure of the lipopeptide cap, dipalmitoyl-S-glyceryl cysteine (Pam2C or Pam2Cys).

FIG. 3B is a chemical structure of the lipopeptide cap, N-acetyl (dipalmitoyl-S-glyceryl cysteine) ((NAc (Pam2C) or NAc(Pam2Cys)).

FIG. 3C is a chemical structure of the lipopeptide cap, tripalmitoyl-S-glyceryl cysteine (Pam3C or Pam3Cys).

### SUMMARY OF THE INVENTION

The compositions and methods described herein are useful as therapeutic and/or prophylactic immunogenic compositions to address this need in the art.

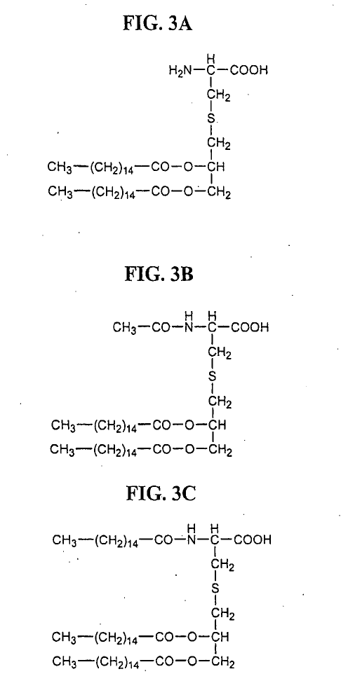

In one embodiment, a self-adjuvanting immunogenic composition useful in the treatment or prophylaxis of Alzheimer's disease is described. This composition includes an immunogen composed of a lipopeptide cap (R2), a universal T helper sequence (R1), and an amyloid β B cell epitope. In one embodiment, each immunogen has the formula: R2−(R1− amyloid β B cell epitope) (Formula I). According to this formula, R2 has three positional locations in the immunogen. In one embodiment, the R2 lipopeptide cap (See FIGS. 3A-3C) is linked, via its Cys or via an optional linker sequence of up to 10 amino acids, to the α-amino group of the amino-terminal amino acid of the T helper sequence R1, which is linked to the amino terminus of the B cell epitope. In another embodiment, the R2 lipopeptide cap is linked via its Cys or its optional linker amino acid(s) to the ε-amino group of a lysine residue inserted between R1 T helper sequence and the B cell epitope. In still another embodiment, the R2 lipopeptide is linked via its Cys or its optional linker amino acid(s) to the ε-amino group of a lysine residue inserted at the C terminus of B cell epitope of the immunogen. In yet other embodiments, the R1 helper sequence and B cell epitope may be in reverse order, with the R2 lipopeptide cap linked via its linker amino acid(s) in any one of three above-noted positions. Still other orders of arrangement of the immunogen components are contemplated, such as by the alternative formulae disclosed herein.

In some embodiments, the R2 lipopeptide cap comprises a linker of one to ten amino acids to link it to the other components of the immunogen. In other embodiments a similar linker is employed to link other components of the immunogen together.

In further embodiments, a sequence of charged, polar amino acids provided with or without the linker sequence is present in various positions in the immunogen. In one embodiment, charged polar sequence is inserted after the R2 cap's Cys, or between the R2 lipopeptide cap's optional linker neutral amino acids and the R1 helper sequence. In other embodiments a linker alone or with such a polar sequence is inserted between R1 helper sequence and the amyloid β B cell epitope, in either order. In still further embodiments, a linker alone or with such a polar sequence is inserted at the free carboxy terminus of R1 or the amyloid β B cell epitope, or the free amino terminus of the R1 or B cell epitope. In still further embodiments, a linker alone or with such a polar sequence is inserted before or after a lysine residue inserted into various embodiments of the immunogen.

In certain embodiments, for each immunogen, R2 is dipalmitoyl-S-glyceryl cysteine (Pam2Cys) of FIG. 3A comprising optionally one or up to ten linker amino acids, as described below. In certain embodiments for each immunogen, R2 is N-acetyl (dipalmitoyl-S-glyceryl cysteine) ((NAc(Pam2C)) of FIG. 3B, which also can comprise an optional amino acid linker. In certain embodiments for each immunogen, R2 is a lipopeptide tripalmitoyl-S-glyceryl cysteine (Pam3Cys) of FIG. 3C, which can optionally comprise a linker sequence. The compositions induce anti-Aβ antibodies with geometric mean titers (GMT) of at least 50,000, at least 300,000 or greater than 1 million, when employed to immunize a mammalian subject.

In another embodiment, a pharmaceutical composition comprises the self-adjuvanting immunogenic compositions defined herein, and a suitable pharmaceutical carrier or excipient. This composition also demonstrates induction of anti-Aβ antibodies with GMT greater than 50,000, or greater than 300,000 or greater than 1 million, when a mammalian subject is immunized therewith.

In yet another embodiment, a method of inducing in vivo the production of anti-Aβ antibodies having a high GMT by immunizing a subject with an effective antibody-inducing amount of the immunogenic or pharmaceutical compositions described herein. In certain embodiments, the GMT is 50,000 or greater than 60,000. In other embodiments, particularly where the method employs a prime dose and one or more booster doses of the composition, the antibodies have a GMT considerably higher, e.g., on the order of greater than 100,000, greater than 200,000, greater than 300,000 or greater than 1,000,000.

In another aspect, use of the immunogens described above in the manufacture of a medicament for the treatment and/or prophylaxsis of Alzheimer's Disease is provided. The medicament induces in vivo the production of anti-amyloid p antibodies with high GMT, even at low dosages.

In yet another aspect, introduction of charged polar residues in at least one position within the immunogen confers aqueous solubility and facilitates an aqueous and/or lyophilized formulation.

Other aspects and advantages of these methods and compositions are described further in the following detailed description.

### DETAILED DESCRIPTION OF THE INVENTION

The compositions and methods described herein address the need in the art for therapeutic and prophylactic compositions and compositions for use in treating, retarding progression of, and preventing, Alzheimer's disease (AD) in human subjects. In one embodiment these compositions involve the formulation and application of therapeutic and/or prophylactic immunogenic therapies that are efficacious against AD without incurring the side effects of meningo-encephalomyelitis, or other adverse reactions related to powerful extrinsic adjuvants. To create such composition, the inventor provides a strict selection of a short Aβ peptide sequence providing an Aβ B cell epitope, but not an Aβ cytotoxic T cell epitope, and couples such a peptide with a powerful immunogenic moiety acceptable for use in humans. By incorporating a sequence that simultaneously enhances a helper T cell response with a B cell response directed to a selected Aβ peptide sequence in combination with a particularly desirable self-adjuvanting lipoprotein, in a single immunogen, the inventor produced a composition that elicits high, persistent levels of anti-Aβ antibody titers in vivo.

### I. Compositions

In one embodiment, the self-adjuvanting immunogenic composition comprises a specifically designed immunogen employing a short Aβ peptide, which enables the compositions to induce anti-Aβ antibodies with geometric mean titers of greater than 50,000, greater than 300,000 or greater than 1,000,000. Each “immunogen” as used herein is a composition that does not occur in nature, but can be produced by synthetic technologies, e.g., chemical synthetic techniques for peptides and lipopeptides. This chemical synthesis is completely scalable, allowing for a relatively inexpensive process for producing large quantities of immunogen. Recombinant DNA preparation and expression may also be employed to construct some portions of the immunogen, at the selection of the person of skill in the art.

In one embodiment, an immunogenic composition comprises an immunogen including a lipoprotein cap (R2), a universal T helper sequence (R1) and a short Aβ protein B-cell epitope. These immunogen components are described in detail below. In a further embodiment, an immunogenic composition comprises an immunogen of

R2−(R1−Aβ B cell epitope), or alternatively Formula I

R2−(Aβ B cell epitope−R1), or alternatively Formula II

R2−K(Aβ B cell epitope)−R1, or alternatively Formula IIIa

R2−K(R1)− Aβ B cell epitope. Formula IIIb

In each Formula I and II, the R2 lipopeptide cap may take one of three positions, as described herein. In one embodiment, the R2 cap is linked to the α-amino of an N-terminal amino acid of either R1 (Formula I) or the B cell epitope (Formula II) via the R2 Cys or its optional linker amino acid(s). In other embodiments of the above formulae, an optional lysine residue (K) is inserted between the R1 T helper sequence and the B cell peptide (Formula I), the B cell peptide and R1 (Formula II), at the C-terminal end of R1 (Formula II) or at the C-terminal end of the amyloid β B cell epitope (Formula I). The R2 is linked to the ε-amino group of the inserted K via the R2 Cys or its optional linker amino acid(s) in these latter embodiments.

In Formulae IIIa, the K is an inserted lysine residue. The carboxy terminus of the B cell epitope within the parentheses is linked to the ε-amino group of the K. The amino terminus of the R1 is lined to the carboxy terminus of the K residue. Similarly, in Formulae IIIb, the K is an inserted lysine residue. The carboxy terminus of R1 within the parentheses is linked to the ε amino group of the K. The amino terminus of the B cell epitope is attached to the carboxy terminus of the K residue.

In certain embodiments, R2 (and optionally R1) individually include an amino acid sequence or linker sequence of from 0 to 10 amino acids in length, which links the lipopeptide of R2 to the other components forming the immunogen, (or links R1 to the B cell epitope) depending upon the formula selected. In other embodiments, a charged polar amino acid sequence is inserted into the immunogen formula with or without flanking linker amino acids, between the components of the formula, at the free amino or carboxy termini of R1 or the B cell epitope or interposed before or after the inserted lysine residue, to enhance solubility. The linker and polar charged sequences are described in detail below.

The immunogens described herein can form a variety of structures, based upon the selection of the formula above. In one embodiment of an immunogen of Formula I, the R2 lipopeptide cap, which contains a Cys and optionally one or more neutral linker amino acids, is linked to an α-amino group at the amino terminus of the R1 T helper sequence, which is linked to the B cell epitope, thus forming a linear construct. An optional polar charged sequence is located after the R2 Cys, or between the R2 linker amino acids, thus linking to R1, but may also be located at additional positions between R1 and the B cell epitope or at the carboxy terminal end of the immunogen to enhance solubility. Immunogens of this formula are described in the examples below. In another embodiment of an immunogen of Formula I, R2 is linked to an ε-amino group of a K residue located between R1 and the first N-terminal amino acid residue of the B cell epitope via the R2 Cys or its optional linker amino acids only and/or via a charged, polar sequence optionally flanked by neutral linker amino acids. In still another embodiment of an immunogen as defined by Formula I, R2 is linked via its Cys, its optional linker amino acid(s) and/or polar charged sequence to an ε-amino group of a K residue located at the C terminus of the B cell epitope.

Still other embodiments of immunogens as described herein can take form of Formula II. In one embodiment of an immunogen of Formula II, the R2 lipopeptide cap with its optional linker amino acid(s) and/or polar charged amino acid sequence, is linked to an α-amino group at the amino terminus of the amyloid ε B cell epitope, which is linked to the R1 helper sequence. In another immunogen of Formula II, the R2 lipopeptide cap is linked via its Cys, its optional linker amino acid(s) and/or its polar charged sequence, or a combination of same, to an ε-amino group of a K residue located between the amyloid β B cell epitope and the first N-terminal amino acid residue of R1. In another embodiment, the R2 lipopeptide cap is linked as described above to an ε-amino group of a K residue located at the C terminus of the R1 in Formula II. Optional linker and/or polar charged sequences may be located between one or more of these immunogen components. Other alternative immunogens may be designed employing these components and the above formulae by one of skill in the art given the teachings of this specification.

### A. The Aβ B Cell Epitope

The Aβ B cell epitope used in the formulae above is a peptide sequence of 7 to 17 Aβ amino acids in length from within the first 17 Aβ amino acids (i.e., amino acids 1-17 of SEQ ID NO: 1), having no T cell epitope. Thus, in one embodiment, the Aβ epitope is Aβ15, having the sequence, in single letter abbreviations for the amino acids: -D-A-E-F-R-H-D-S-G-Y-E-V-H-H-Q- SEQ ID NO: 2. In another embodiment, the Aβ B cell epitope is Aβ10, having the sequence of -D-A-E-F-R-H-D-S-G-Y-, amino acids 1-10 of SEQ ID NO: 2.

In other embodiments, the Aβ B cell epitope of the immunogens forming the composition contains all or less than all of the first 16 or first 15 amino acid residues of Aβ. Thus the Aβ epitope in one embodiment is Aβ16 or Aβ15 or Aβ14 or Aβ13 or Aβ12, or Aβ11 or Aβ10. In other embodiments, the Aβ epitope is Aβ2-10, Aβ2-11, Aβ2-12, Aβ2-13, Aβ2-14, Aβ2-15, or Aβ2-16. In other embodiments the epitope is Aβ3-10, Aβ3-11, Aβ3-12, Aβ3-13, Aβ3-14, Aβ3-15, or Aβ3-16. In still another embodiment, the Aβ epitope is Aβ3-10. The Aβ epitope may have additional amino acid residues at the C terminus to enhance immunogenicity, but may never incorporate a cytotoxic T cell epitope. For ease of discussion and exemplification, references to the Aβ B cell epitope will hereafter refer to the embodiment Aβ15 (i.e., β amyloid amino acid residues 1-15) or Aβ10 (i.e., P amyloid amino acid residues 1-10). However, those of skill in the art realize that the embodiments are not limited to that epitope.

It is also possible to modify individual amino acid residues in the Aβ B cell epitopes described above. For example, other Aβ B cell epitopes may include modifications at one or more amino acid residues of the epitopes specifically described above. For example, a naturally-occurring amino acid of the Aβ B cell epitope may be conservatively replaced individually by amino acid residues having similar characteristics. For example, an amino acid residue of Aβ15 or Aβ10 may be replaced by other amino acid residues bearing the same charge and/or similar side chain lengths. Similarly a naturally-occurring amino acid in Aβ15 or Aβ10 may be replaced by unnatural amino acid residues, i.e., an amino acid having a modification in the chemical structure, e.g., a D-amino acid, an amino acid bearing a non-naturally occurring side chains an N-methylated amino acid, etc. See, the cited references relating to N-methylated amino acids, among others. See, e.g., L. Aurelio et al, 2002 Organic Letters, 4(21):3767-3769 and references cited therein.

Further, longer peptides incorporating the Aβ amino acid residues of 1-15 or 1-10 but containing additional amino acids at the N and/or C termini, may be employed, although such longer sequences are unlikely to provide any functional difference to the immunogens. In other embodiments, other amyloid β B cell epitope peptides may contain smaller sequences of about 6 of such amino acids to about 25 amino acid residues in length. However, there may be no perceptible advantage to the use of any epitope other than the exemplified Aβ15 or Aβ10.

In certain embodiments of immunogens of Formula II, the N terminus of the Aβ epitope is free and only the C terminus is coupled to another component of the immunogen.

In yet another embodiment, an alternative immunogen may be prepared for use with the amyloid β epitope immunogens described herein. See, for example, the description of alternative immunogens in Example 1 below. The use of such alternative immunogens may enhance the response induced by the immunogenic compositions containing the B cell epitope immunogens described herein.

### B. The Universal T Helper Sequence R1

Another component of the immunogens of the immunogenic compositions described herein is a universal T helper epitope used to enhance the immunogenicity of the B cell epitope in the immunogen, e.g., the Aβ15 or Aβ10 epitope. The term “T helper epitope” is intended to mean a chain of amino acids which, in the context of one or more class II MHC molecules, activates T helper lymphocytes, which enhances the antibody response to the Aβ15 or Aβ10 peptide of the immunogen. In certain embodiments, the T helper epitope component of the immunogens is one that is recognized by T helper cells present in the majority of the population. This can be accomplished by selecting peptides that bind to many, most, or all of the HLA class II molecules. These are known as “loosely HLA-restricted” or “promiscuous” T helper sequences. In particular, promiscuous T cell epitopes are used as the R1 moiety in the formulae above.

Also, depending upon the formula of the immunogen selected, the linkage between R1 and R2 is one of the following: R2 via its Cys or its optional linker amino acid(s) and/or polar charged sequence, is linked to the α-amino of the N terminal amino acid of R1. Alternatively R2 is linked via its Cys or its optional linker amino acid(s) and/or polar charged sequence, to the ε amino group of an additional lysine residue inserted between R1 and the B cell epitope. Still alternatively if R1 is located at the carboxy terminus of the immunogen, R2 may be linked via its Cys or its optional linker amino acid(s) and/or polar charged sequence, to an e-amino group of a lysine inserted at the carboxy terminus of R1. In Formula IIIa, R2 is linked via its Cys or its optional linker amino acid(s) and/or polar charged sequence, to the inserted K residue, which is linked to the N terminal amino acid of R1, and the B cell epitope is linked to the ε-amino group of the K. In Formula IIIb, the R2 is linked via its Cys or linker/polar sequences to an inserted K, the carboxy terminus of the R1 is linked via the ε amino group of the inserted K, and the B cell epitope is linked via its N terminus to the inserted K.

Many promiscuous or universal T helper sequences occur naturally in different sources, e.g., microorganisms, or are artificially engineered sequences. Such suitable T cell epitopes are known and may be selected for this use in these immunogens and compositions.

In one embodiment, and as exemplified by the examples below, the R1 T helper sequence in an immunogen as described in Formula I, II, IIIa and/or IIIb herein has the sequence, Q-Y-1-K-A-N-S-K-F-I-G-I-T-E-Xaa SEQ ID NO: 3, wherein said Xaa is absent or L. This sequence is naturally found in the tetanus toxin at amino acids 830-843(844); see, Panina-Bordignon et al. 1989 Eur J Immunol 19:2237. Another such tetanus toxin sequence (aa 947-967 of tetanus toxin) useful as R1 has the sequence F-N-N-F-T-V-S-F-W-L-R-V-P-K-V-S-A-S-H-L-E SEQ ID NO: 4, or a derivative thereof, such as aa950-969 of Tet toxoid. See, Reece J C et al. 1994 J Immunol Methods 172:241-54. Still other tetanus toxoid T cell helper sequences for use as the R1 of the formulae include the sequences I-D-K-I-S-D-V-S-T-I-V-P-Y-I-G-P-A-L-N-I SEQ ID NO: 5, aa632-651 of Tet toxoid, N-S-V-D-D-A-L-I-N-S-T-K-I-Y-S-Y-F-P-S-V SEQ ID NO: 6, aa580-599 of Tet toxoid, P-G-I-N-G-K-A-I-H-L-V-N-N-E-S-S-E SEQ ID NO: 7, aa 916-932 of Tet toxoid, and Z-Y-I-K-A-N-S-K-F-I-G-I-T-E SEQ ID NO: 8, aa 830-842 of Tet toxoid. For still other universal T cell helper sequences useful as the R1 of the immunogens, see, e.g., Ho et al. 1990 Eur J Immunol 20:477; Valmori et al 1992 J Immunol 149:717-721; Chin et al. 1994 Immunol 81:428, Vitiello et al 1995 J Clin Invest 95:341; Livingston et al. 1997 J Immunol 159:1383; Kaumaya P T P et al. 1993 J Mol Recognition 6:81-104 (1993), and Diethelm-Okita B M et al. 2000 J Inf Dis 175:383-91; all incorporated by reference herein. See also, Raju et al. 1995 Eur J Immunol 25:3207-14 and Diethelm-Okita B M et al. 2000 J Inf. Dis 181:1000-9, incorporated by reference herein, which discuss certain diphtheria toxin T cell helper sequences which may be employed as R1 in the immunogens described herein. Still other sequences which may be useful as T helper epitope sequences for R1 of the formulae above are disclosed in Nardin et al. 2001 J Immunol 166:481-10, incorporated by reference herein.

Examples of other T helper sequences that are promiscuous include sequences from antigens such as Plasmodium falciparum circumsporozoite (CS) protein at positions 378-398 (D-I-E-K-K-I-A-K-M-E-K-A-S-S-V-F-N-V-V-N-S; SEQ ID NO: 9), and Streptococcus 18 kD protein at positions 116 (G-A-V-D-S-1-L-G-G-V-A-T-Y-G-A-A; SEQ ID NO: 10). See, e.g., U.S. Pat. No. 7,026,443, incorporated herein by reference.

The R1 universal T cell helper sequence may also be an artificially engineered sequence, such as the Pan HLA DR-binding (PADRE) molecule (Epimmune, San Diego, Calif.) described, for example, in U.S. Pat. No. 5,736,142 (see, e.g., PCT publication WO 95/07707, incorporated by reference herein). These synthetic compounds are designed to most preferably bind most HLA-DR (human HLA class II) molecules. Other examples include peptides bearing a DR 1-4-7 supermotif, or either of the DR3 motifs. These sequences are recognized by class 2 mixed histocompatibility (MHC) antigens on B cells and macrophages and dendritic cells and enhance B cell production of antigens.

Thus, in one embodiment, the R1 sequence is defined by the formula Xaa1-K-Xaa2-V-A-A-W-T-L-K-A-A-Xaa3 SEQ ID NO: 11, wherein Xaa1 and Xaa3 are independently selected from D-Alanine or L-Alanine, and Xaa2 is L-cyclohexylalanine, phenylalanine, or tyrosine. These T helper sequences have been found to bind to most HLA-DR alleles, and to stimulate the response of T helper lymphocytes from most individuals, regardless of their HLA type. In one embodiment, R1 has the above formula, in which Xaa1 and Xaa3 are both D-Alanine and Xaa2 is cyclohexylalamine. Other PADRE sequences include an alternative of a pan-DR binding epitope that comprises all “L” natural amino acids and can be provided in the form of nucleic acids that encode the epitope. Still other PADRE sequences are disclosed in Vitiello et al. 1995 J Clin Invest 95:341; Alexander J et al. 1994 Immunity 1:751-61; Del Guercio M-F et al. 1997 Vaccine 15:441-8; Alexander J et al. 2000 J Immunol 164:1625-33; Alexander J et al. 2004 Vaccine 22:2362-7; and Agadjanyan M G et al. 2005 J Immunol 174:1580-6, all incorporated by reference herein.

These T helper peptide sequences R1 can also be modified to alter their biological properties. For example, they can be modified to include D-amino acids or other amino acid modifications to increase their resistance to proteases and thus extend their serum half life. Further these promiscuous T cell helper sequences or R1 sequences may further include linker and/or polar charged sequences as discussed below.

One of skill in the art is expected to select from among other known promiscuous T cell helper sequences to design other specific immunogens for immunogenic compositions as described herein. A specific embodiment, described below, illustrates two universal T helper sequences that have been useful within the immunogens at inducing antibodies with high geometric mean titers (GMT) against amyloid β protein.

### C. The Lipopeptide Cap Component R2

Another component of the immunogens described herein is a lipid component, preferably a “lipopeptide cap”, to work, in concert with the other components of the immunogens to induce antibodies with GMT of greater than 50,000, or greater than 60,000, or greater than 300,000 or greater than 1,000,000, needed for the prophylactic and therapeutic immunogenic compositions as described herein. Lipopeptides have been identified as agents capable of priming CTL and also enhancing humoral antibody responses in vivo against certain antigens. Thus, the R2 moiety of the immunogens is selected from among desirable lipopeptide components having attached thereto a Cys and optionally from one up to ten amino acid linker residues and/or optionally a sequence of charged polar amino acids as described below.

In one embodiment, the R2 lipopeptide is attached directly or via its optional linker and/or polar charged amino acid(s) to an α-amino group at the amino terminus of the immunogen, i.e., it is attached to the amino terminus of the R1 T cell helper sequence or directly to the Aβ B cell epitope, if the R1 is in a different position. In another embodiment of an immunogen, the R2 lipopeptide is attached directly via its Cys or via its optional one up to ten neutral amino acid linker residues and/or optionally a sequence of charged polar amino acids to an e-amino of a K residue located between R1 and the first N terminal amino acid residue of the Aβ B cell epitope. In yet a further embodiment, the immunogen's R2 lipopeptide cap is linked directly via its Cys or via its optional one up to ten neutral amino acid linker residues and/or optionally a sequence of charged polar amino acids to an ε-amino of a K residue located at the C-terminus of the immunogen, i.e., the C-terminus of the Aβ15 or Aβ10 epitope or R1. In yet a further embodiment, the immunogen's R2 lipopeptide cap is linked directly via its Cys or via its optional one up to ten neutral amino acid linker residues and/or optionally a sequence of charged polar amino acids directly to a K residue, which is linked directly to the N-terminus of either the B cell epitope or the R1. In this structure, either the B cell epitope or the R1 may be linked via its carboxy terminus to the ε amino group of the K (see Formulae IIIa or IIIb).

Specific R2 lipopeptides for such use include, e.g., N-terminal sequences of the E. coli lipoproteins. In one embodiment, R2 is a lipopeptide which is dipalmitoyl-S-glyceryl cysteine (Pam2Cys) of FIG. 3A with two amino acid linkers and/or a polar charged sequence. In another embodiment, R2 is a lipopeptide which is tripalmitoyl-S-glyceryl cysteine (Pam3Cys) of FIG. 3C with its amino acid linker and/or polar charged sequence.

Other R2 caps include an R-(dipalmitoyl-S-glyceryl) cysteine, wherein the R is a group consisting of a hydrogen, an alkyl, alkenyl or alkynl of 1-6 C atoms. In one embodiment, R2 is a lipopeptide which is N-acetyl (dipalmitoyl-S-glyceryl cysteine) (NAc(Pam2C)) of FIG. 3B with an optional amino acid linker and/or polar charged sequence (R is N-acetyl). Other potential R2 moieties include hexadecanoic acid, Hda, and macrophage activating peptide, and MALP-2.

Such lipopeptide caps may be selected, synthesized and prepared from those described by Deres, et al., 1998 Nature 342:561; Weismuller et al. 1989 Vaccine 7:29; Metzger et al. 1991 Int Peptide Protein Res 38:545; Martinon et al. 1992 J Immunol 149:3416; Vitiello et al. 1995 J Clin Invest 95:341; Muhlradt et al. 1997 J Exp Med 185:1951; Livingston et al. 1999 J Immunol 162:3088; Zeng et al 2002 J Immunol 169:4905; Borzutsky et al. 2003 Eur J Immunol 33:1548; Scgjetne et al. 2003 J Immunol 171:32; Jackson et al. 2004 Proc Natl Acad Sci USA 101:15440 Borzutsky et al. 2005 J Immunol 174:6308; Muhlradt P F et al. J Exp Med 11:1951-8 (1997); Obert M et al. Vaccine 16:161-10 (1997); Zeng W et al. Vaccine 18:1031-10 (2000); Gras-Masse H Mol Immunol 38:423-31 (2001); Zeng W et al. J Immunol 169:4905-12 (2002); Schjetne K W et al. J Immunol 171:32-6 (2003); Spohn R et al. Vaccine 22:2494-9 (2004); Jackson D C et al. Proc Natl Acad Sci USA 101:15440-5 (2004); Zeng W et al. Vaccine 23:4427-35 (2005); International Patent Application Publication Nos. WO2006/026834, WO2006/040076, WO2004/014956 or WO2004/014957, all above-cited documents incorporated by reference herein.

In one embodiment, a particularly effective immunogenic composition comprises the R2 of Pam2CSS, i.e., the dipalmitic acid moiety dipalmitoyl-S-glyceryl-Cys, which is attached via a linker, e.g., Ser-Ser, and/or a polar charged sequence. A Pam2C with a linker is described in PCT publication WO 2004/014957, incorporated herein by reference. In another embodiment, a particularly effective immunogenic composition comprises the R2 of Pam3Cys-S-S-, i.e., the tripalmitic acid moiety dipalmitoyl-S-glyceryl-Cys, which is attached via a linker, e.g., Ser-Ser, and/or a polar charged sequence. In another embodiment, the R2 is NAc(Pam2C)-S-S-, i.e., the dipalmitic acid moiety N-acetyl (dipalmitoyl-S-glyceryl cysteine), which is attached via a linker, e.g., Ser-Ser, and/or a polar charged sequence.

As previously described, this R2 lipopeptide is linked directly via its Cys or via its optional one up to ten amino acid linker residues and/or optionally a sequence of charged polar amino acids, to an α-amino group at the amino terminus of the R1 T cell helper sequence, which is in turn linked to the B cell epitope. If the B cell epitope and T helper sequence are reversed, as in Formula II, this R2 lipopeptide is linked, via its Cys, or its optional linker and/or optional polar charged sequence, to an α-amino group at the amino terminus of the B cell epitope, which is in turn linked to the R1. Alternatively R2 is linked via its Cys, its suitable linker amino acid(s) and/or polar charged amino acid sequence, to an ε-amino of a K residue located between R1 and the first N terminal amino acid residue of the B cell epitope component of the immunogen. This same structure is duplicated if the R1 and B cell epitope are reversed, as in Formula II. In still another alternative immunogen structure, the R2 is linked via its Cys, its optional linker amino acid(s) and/or polar charged amino acid sequence to an 1-amino of a K residue inserted at the C terminus of the immunogen. For instance, if the R1 is linked to the B cell epitope, a lysine residue may be inserted at the C-terminus of the B cell epitope and R2 linked to the E-amino group of that lysine via a linker. This structure is similar if the B cell epitope and R1 group are reversed, as in Formula II. Similarly immunogens of the Formulae IIIa and IIIb are as described above using this R2 cap. In all embodiments, the N-terminal end of the R2 lipopeptide cap is free and not bound to another component of the immunogen.

The R2 cap enhances the antibody response, and proves highly effective in the immunogens of Formula I, II, IIIa and/or IIIb that form the immunogenic compositions. Still other embodiments of the variety of attachments of the R2 to the R1 and/or the B cell epitopes of the immunogens are embodied in the Formulae I, II, IIIa and IIIb described above.

### D. The Optional Linkers and Polar Sequences

Although the R2 cap can be directly linked through its Cys, and the T cell helper sequences R1 can be directly linked to the B cell epitope component of the immunogen in either order, a linker is desirably optionally incorporated to link the C-terminal end of the R2 lipopeptide cap component to any other component in each immunogen. In other embodiments, linker amino acids or sequences are used also between the B-cell epitope and the R1 helper sequence. In another embodiment, an amino acid sequence is used as an optional linker attached to the N- or C-termini of the R1 helper sequence or the B cell peptide to couple one component to another component of the immunogen, depending upon the formula of the immunogen.

The “linker” located within the R2 cap or positioned elsewhere in the immunogen is typically comprised of from one to ten relatively small, neutral molecules, such as amino acids or amino acid mimetics, which are substantially uncharged under physiological conditions. The linkers are typically selected from, e.g., Gly, Ser, Pro, Thr, or other neutral linkers of nonpolar amino acids or neutral polar amino acids. The optional linker need not be comprised of the same residues and thus may be a heterooligomer, e.g., Gly-Ser- or a homooligomer, e.g., Ser-Ser. When present, the linker in one embodiment is at least one amino acid residues, e.g., Ser or Gly. In another embodiment, the linker is at least two amino acid residues, e.g., Ser-Ser or Gly-Ser. In still other embodiments three to six amino acid residues, and up to 10 or more residues are used to form the linker. Thus in certain embodiments, the linker sequence includes at least 1, 2, 3, 4, 5, 6, 7, 8, 9 or 10 amino acids or mimetics.

For example, a linker may be used to couple the lipopeptide cap of R2 to another component of the immunogen. In one embodiment, the linker is the dipeptide Ser-Ser. In another embodiment, the linker is Gly-Gly. In still another embodiment, a heterooligomer, such as Gly-Ser, or Ser-Gly may be used. In another embodiment, a linker such as -Ser-links the T helper sequence R1 to the N-terminal amino acid of the B cell epitope in the immunogen or links the B cell epitope to the N-terminal amino acid of the R1.

In another embodiment of the immunogens, a sequence of charged, polar amino acids is incorporated within, or replaces, the relatively uncharged linker sequences. Introduction of a charged, polar sequence has been found to enhance the aqueous solubility of the composition, as demonstrated by the examples below. For example, these charged polar sequences are employed to enhance the solubility of the immunogens in formulation with water for injection and optionally mannitol for tonicity, without the need for a buffer. These charged polar sequences enable the immunogens to be readily prepared, solubilized and lyophilized. These polar sequences, by enhancing solubility, may also be useful to enhance the immunogenicity of the B cell epitope.

In one embodiment, the polar sequence is composed of 4, 5, 6, 7, or 8 charged polar amino acids. In a further embodiment, the polar sequence is composed of 4 amino acids. In yet another embodiment, the polar sequence is composed of 6 amino acids. In one embodiment, the polar sequence is composed of amino acids selected from lysine, arginine, aspartate, and glutamate. In a further embodiment, the polar sequence is composed of amino acids selected from lysine, arginine, and aspartate. In another embodiment, the amino acids in the polar sequence are identical. In further embodiments, 2, 3, or 4 different amino acids are used in the polar sequence. Thus, in one embodiment, a polar, charged sequence is -Lys-Lys-Lys-Lys-, (SEQ ID NO: 35) -Lys-Lys-Lys-Lys-Lys-Lys-, (SEQ ID NO: 36) or -Lys-Glu-Lys-Glu- (SEQ ID NO: 37) or -Glu-Glu-Glu-Glu- (SEQ ID NO: 38) or any iteration of from 4 to 8 identical or varying polar, charged amino acids.

Optionally, the polar amino acid sequence is flanked on either terminus by an amino acid of the linker to form the sequence-linker amino acid-(polar amino acid)ₙ-linker amino acid-, wherein n is the number of polar amino acids, e.g., from 4 to 8. Alternatively, the polar amino acid sequence may be used without the flanking linker (neutral, uncharged) amino acids. In one embodiment, the linker with a polar amino acid sequence is composed of Ser-Lys-Lys-Lys-Lys-Ser, (SEQ ID NO: 39) i.e., a 4 identical amino acid polar sequence within a Ser-Ser linker, or Ser-[Lys]₄-Ser (SEQ ID NO: 39). In another embodiment, the amino acid linker containing a polar sequence is Ser-[Lys]₆-Ser (SEQ ID NO: 40). In other embodiments, the linker with polar sequence is Gly-[Lys]₄-Gly (SEQ ID NO: 41) or Gly-[Lys]₆-Gly (SEQ ID NO: 42). In still other embodiments the linker with polar sequence is -Ser-(Lys-Glu-Lys-Glu-)-Ser- (SEQ ID NO: 43). As above, any iteration of this sequence that can be assembled by one of skill in the art given the above definition and the—linker amino acid-(polar amino acid)ₙ-linker amino acid—formulae.

In one specific embodiment, therefore, an amino acid linker containing a polar, charged sequence, or the linker alone, or the polar charged sequence alone, is located between the immunogen component R2 and any other component of the immunogen with which it is linked. In another embodiment, the linker and/or polar sequence is located between R1 and any other component of the immunogen. In still another embodiment, the linker and/or polar sequence is located between the B cell epitope and any other component of the immunogen. In another embodiment, a polar sequence may be attached with or without flanking linker amino acids to the free terminus of the B cell epitope or R1, i.e., external to the immunogen, such as to the free N- or C-terminus of a terminal B cell epitope or R1 helper sequence in the immunogen.

In another embodiment, a polar charged sequence with or without flanking linker amino acids is present only once in the immunogen, e.g., attached only to the carboxy-terminal linker-Ser- of the di- or tripalmitic acid moiety of R2, linking R2 to R1 or the B cell epitope or to an inserted lysine in the immunogen. In still another embodiment, linker and/or polar sequences are present in multiple (i.e., 2 or more) positions in the immunogen.

The R1, R2 and B cell epitope components of each immunogen forming the immunogenic composition can also be modified by the addition of linker amino acids and/or polar charged sequences to the termini of each component to provide for coupling to a carrier support or larger peptide, for modifying the physical or chemical properties of the peptide or oligopeptide, or the like.

### E. Specific Embodiments

Specific embodiments of immunogens of Formula I, II, IIIa or IIIb are employed in the examples below and also include the following immunogens. In one embodiment, in each immunogen, R2 is formed by two units of the palmitic acid linked via a thiolglyceryl group to a cysteine and an amino acid linker sequence of -S-S- residues, which links the R2 to the first amino acid of R1. R1 is Q-Y-I-K-A-N-S-K-F-I-G-I-T-E-L SEQ ID NO: 12 with an optional amino acid linker of -S-, which links to the N-terminal amino acid residue of the Aβ15 or Aβ10 peptide. Thus one immunogen of Formula I is defined as follows:

SEQ ID NO: 13

Pam2C-S-S-Q-Y-I-K-A-N-S-K-F-I-G-I-T-E-L-D-A-E-F-R-
H-D-S-G-Y-E-V-H-H-Q-amide.

Another immunogen of Formula I is defined as follows:

SEQ ID NO: 14

Pam2C-S-S-Q-Y-I-K-A-N-S-K-F-I-G-I-T-E-L-D-A-E-F-R-
H-D-S-G-Y-amide.

In a further embodiment, a polar sequence of four lysines (underlined) is incorporated in the linker sequence (-S-S-) connecting R2 to R1. Such further immunogens of Formula I are defined as follows:

Pam2C-S-K-K-K-K-S-Q-Y-I-K-A-N-S-K-F-I-G-I-T-E-L-D-A-E-F-R-H-D-S-G-Y-E-V-H-H-Q- amide SEQ ID NO: 30. Similar immunogens employ the shorter Aβ10 sequence for the B cell epitope in this construct.

In still a further embodiment, a linker containing a polar sequence of six lysines (underlined) is utilized to link R1 helper sequence to the B cell epitope component. This further immunogen of Formula I is defined as follows:

Pam2C-S-S-Q-Y-I-K-A-N-S-K-F-I-G-I-T-E-L-S-K-K-K-K-K-K-S-D-A-E-F-R-H-D-S-G-Y-E-V-H-H-Q-amide SEQ ID NO: 31. Similar immunogens employ the shorter Aβ10 sequence for the B cell epitope in this construct.

In yet a further embodiment, a linker containing a polar sequence of four lysines (underlined) is utilized in two places in the immunogen, i.e., both as part of the R2 linker to link the lipopeptide to the remainder of the sequence and to link R1 to the B cell epitope component. This further immunogen of Formula I is defined as follows:

Pam2C-S-K-K-K-K-S-Q-Y-I-K-A-N-S-K-F-I-G-I-T-E-L-S-K-K-K-K-S-D-A-E-F-R-H-D-S-G-Y-E-V-H-H-Q-amide SEQ ID NO: 32. Similar immunogens employ the shorter Aβ10 sequence for the B cell epitope in this construct.

In yet a further embodiment, a linker containing a polar sequence of six lysines (underlined) is utilized in two places in the immunogen, i.e., both as part of the R2 linker to link the lipopeptide to the remainder of the sequence and at the carboxy terminus of the immunogen. This further immunogen of Formula I is defined as follows:

Pam2C-S-K-K-K-K-K-K-S-Q-Y-I-K-A-N-S-K-F-I-G-I-T-E-L-D-A-E-F-R-H-D-S-G-Y-E-V-H-H-Q-S-K-K-K-K-K-K-S-amide SEQ ID NO: 33. Similar immunogens employ the shorter Aβ10 sequence for the B cell epitope in this construct.

In one embodiment, in each immunogen, R2 is formed by two units of the palmitic acid linked via a glyceryl group to the sulfur of an N-acetyl-cysteine (see FIG. 3B) and an amino acid linker sequence of -S-S- residues, which links the R2 to the first amino acid of R1. R1 is Q-Y-I-K-A-N-S-K-F-I-G-I-T-E-L SEQ ID NO: 12 with an amino acid linker of -S-, which links to the N-terminal amino acid residue of the Aβ15 or Aβ10 peptide. Thus one immunogen of Formula I is defined as follows:

NAc(Pam2C)-S-S-Q-Y-I-K-A-N-S-K-F-I-G-I-T-E-L-D-A-E-F-R-H-D-S-G-Y-E-V-H-H-Q-amide SEQ ID NO: 29. Similar immunogens employ the shorter Aβ10 sequence for the B cell epitope in this construct. In further embodiments, polar sequences are encompassed parallel to the Pam2C containing embodiments reflected above.

In another embodiment, in each immunogen as defined above, R2 is formed by two units of palmitic acid linked via an -S-glyceryl group to a palmityl-cysteine (i.e., Pam3C in place of Pam2C above) and an amino acid linker sequence of -S-S- residues. In further embodiments in which R2 is Pam3C, polar sequences are encompassed parallel to the Pam2C containing embodiments reflected above.

In an embodiment in which the Pam3CSS- or Pam2CSS- or NAc(Pam2C)-SS-containing moiety is coupled to the εamino of a lysine inserted between the universal T helper sequence and the Aβ15 or Aβ10 epitope, an immunogen SEQ ID NO: 15 is defined as follows:

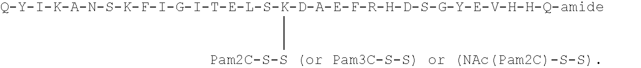

Similar immunogens employ the shorter Aβ10 sequence for the B cell epitope in this construct. In further embodiments, polar sequences are encompassed parallel to the Pam2C containing embodiments reflected above.

In an embodiment in which the Pam3CSS- or Pam2CSS- or NAc(Pam2C)-S-S-containing moiety is coupled to the ε amino of a lysine inserted at the carboxy terminus of the Aβ15 or Aβ10 peptide, an immunogen SEQ ID NO: 16 is defined as follows:

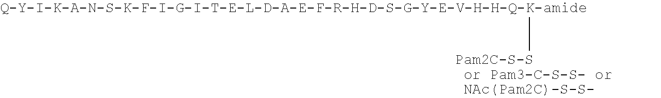

Similar immunogens employ the shorter Aβ 10 sequence for the B cell epitope in this construct. In further embodiments, polar sequences are encompassed parallel to the Pam2C containing embodiments reflected above.

In an embodiment in which the Pam3CSS- or Pam2CSS- or NAc(Pam2)CSS-containing moiety is coupled to the ε-amino of a lysine inserted between the Aβ epitope and the universal T helper sequence, a Formula II immunogen SEQ ID NO: 17 is defined as follows:

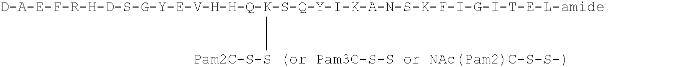

Similar immunogens employ the shorter Aβ10 sequence for the B cell epitope in this construct. In further embodiments, polar sequences are encompassed parallel to the Pam2C containing embodiments reflected above.

In another embodiment of Formula III, in which the Pam3CSS- or Pam2CSS- or NAc(Pam2C)-S-S-containing moiety is coupled via an inserted lysine and serine spacer to the N-terminal amino acid of the R1 helper sequence, and the Aβ15 or Aβ10 epitope is coupled via the ε amino of the same lysine residue, the immunogen SEQ ID NO: 18 is defined as follows:

Pam2C-S-S-K(D-A-E-F-R-H-D-S-G-Y-E-V-H-H-Q-)-Q-Y-I-
K-A-N-S-K-F-I-G-I-T-E-L-amide.

Still other embodiments of the immunogen employ the formula of IIIb, and also employ the shorter B epitope, e.g., Aβ10. In further embodiments, polar sequences are encompassed parallel to the Pam2C containing embodiments reflected above.

Immunogenic compositions that contain an immunogen as above defined and as illustrated in the examples below, are characterized by the ability to induce in mammalian animals Aβ peptide antibodies with a geometric mean titer of 50,000 or 60,000 or greater, as discussed in more detail below. In other embodiments the GMTS are greater than 300,000 or greater than 1,000,000, or greater than 3,000,000.

Given the above teachings, one of skill in the art may readily design other immunogens meeting the formulae by selecting from among the components described above.

### I. Methods of Making the Immunogens and Immunogenic Compositions

Immunogens may be prepared according to the formula above by carrying out a chemical synthesis in solid phase or in solution. Both synthesis techniques are well known to those skilled in the art. For example, such techniques are described in conventional texts such as Atherton and Shepard in “Solid phase peptide synthesis” (IRL press Oxford, 1989), Stewart & Young, SOLID PHASE PEPTIDE SYNTHESIS, 2D. ED., Pierce Chemical Co., 1984) and by Houbenweyl in “Methoden der organischen Chemie” [Methods in Organic Chemistry] published by E. Wunsch Vol. 15-I and II, Stuttgart, 1974, and also in the following articles, which are entirely incorporated herein by way of reference: P E Dawson et al. (Science 1994; 266(5186):776 9); G G Kochendoerfer et al. (1999; 3(6):665 71); et P E Dawson et al., Annu. Rev. Biochem. 2000; 69:923-60. Various automated or computer-programmable synthesizers are commercially available and can be used in accordance with known protocols. Further, individual peptide epitopes can be joined using chemical ligation to produce larger peptides that are still within the bounds of the immunogens of Formula I, II, IIIa and/or IIIb as described herein.

Desirably the synthesis involves building the immunogens in direction from the C terminal towards the N-terminal by first immobilizing the C-terminal-most amino acid residue of the R1 or Aβ peptide on a solid support, such as by using Fmoc chemistry using HBTU on RAMAGE resin. The lipopeptide cap is then synthesized as a lipoamino acid essentially as described in PCT publication NO. WO2004/014957, incorporated herein by reference. For example, in one embodiment, the Pam2Cys or NAc(Pam2C) or Pam3Cys lipopeptide cap is introduced onto the synthetic Aβ epitope sequence and T helper sequence by covalently attaching each lipopeptide moiety directly or indirectly via an optional linker and/or polar charged sequence to an alpha-amino group of the N-terminal amino acid of the T helper sequence or to the Aβ peptide, or to the epsilon amino group of a lysine introduced between the R1 and the Aβ15 or Aβ 10 peptide (in either order) or to the epsilon amino group of a lysine introduced at the C terminus of the Aβ15 or Aβ10 peptide sequence. The resulting immunogen is then cleaved from the resin using standard methods, e.g., trifluoroacetic acid (Reagent K), and optionally converted to a salt, also using conventional methodologies, e.g., a BIO-RAD acetate resin.

The resulting composition contains an immunogen having the lipopeptide cap attached to an α-amino group of the N-terminus of R1 or the B cell epitope, or to an epsilon amino group of a lysine residue inserted between R1 and the Aβ epitope, or to a lysine residue inserted at the C-terminus of R1 or Aβ epitope. If more than one immunogen is present in a composition, each immunogen can have a different R1 helper. A composition can contain immunogens having the lipopeptide cap at a position different from other immunogens in the composition, such as described in Formula I, II, IIIa and/or IIIb. In one embodiment the Aβ15 or Aβ10 peptide is sufficient as the B cell epitope in all immunogenic compositions described herein. The optional linker and/or polar charged sequences can be inserted at various positions in the immunogen as described above using this technique.

An exemplary synthesis is described in Example 1. The immunogenic composition can be minimally purified to remove solvents and reagents. Rigorous purification is not likely to be necessary for the compositions to be safe and efficacious. This synthesized composition is tested in animals, or used in humans, in a partially purified form. However, optional conventional purification schemes may be employed, if necessary.

While the above described synthetic method is preferred for its simplicity, an alternative method of preparing the immunogens involves the use of recombinant DNA technology. As well known in the art, a nucleotide sequence (which encodes the Aβ peptides optional linkers (with or without polar sequences, e.g., for solubility), T cell helper sequences, and linkers for the lipopeptide cap is inserted into an expression vector, transformed or transfected into an appropriate host cell and cultivated under conditions suitable for expression. These procedures are generally known in the art, as described generally in Sambrook et al., MOLECULAR CLONING, A LABORATORY MANUAL, Cold Spring Harbor Press, Cold Spring Harbor, N.Y. (1989). Because one cannot make the Pam2Cys or Pam3Cys lipopeptide caps recombinantly, the subsequent attachment of the lipopeptide cap to any of these constructs involves synthetic methods as described above.

One of skill in the art may readily generate a variety of immunogenic compositions containing an immunogen by following these methods. Similarly, if different immunogens are desired in the composition, the various components of the immunogen of Formula I, II, IIIa and/or IIIb are modified individually or collectively, as provided above, such as modifications to individual amino acids, uses of optional linker sequences, uses of different T cell helper or lipopeptide caps, uses of larger or smaller Aβ peptides, or combinations of different immunogenic compositions made with such modified sequences.

Such immunogenic compositions are able to induce a protective immune response or therapeutic immune response to the Aβ15 or Aβ10 peptides in vivo by inducing anti-Aβ15 or Aβ10 antibodies with geometric mean titers (GMT) sufficient to prevent or at least partially arrest or retard progression of existing Aβ symptoms. The titer is the reciprocal of the greatest serum dilution that is still detected at a level of mean+8 standard deviations (SDs) of control values. The geometric mean titer (GMT) is determined by converting each titer of two or more sera to log₁₀, and averaging these log₁₀ values. The anti-log of this latter value is the GMT. In most embodiments, the GMT is determined from three or more individual titers. The use of the GMT rather than individual titers minimizes extreme outlying results and thus improves accuracy.

In one embodiment, an immunogenic composition as described above induces anti-Aβ antibodies with a GMT of 50,000 or greater. In another embodiment immunogenic compositions induce anti-Aβ antibodies in vivo with GMT of 60,000 or greater. In another embodiment immunogenic compositions induce anti-Aβ antibodies in vivo with GMT of 100,000 or greater. In other embodiments, the titers induced are greater than 150,000. In still other embodiments immunogenic compositions induce anti-Aβ antibody with GMT of 200,000. In other embodiments, the antibodies induced have titers that are greater than 500,000. In still other embodiments immunogenic compositions induce anti-Aβ antibody with GMT of greater than 1,000,000.

The examples below report experiments in laboratory animals that provide evidence of the antibodies with desirably high titers induced by immunogenic compositions described herein. Depending upon the selection and composition of other components used in the pharmaceutical compositions and the regimens and routes of administration of these compositions, the induction of such high titer antibody responses is anticipated with compositions other than those specifically exemplified.

### III. Pharmaceutical Compositions and Methods of Treatment/Prophylaxis

A pharmaceutical composition containing the above-described immunogens is useful for the therapeutic treatment of AD and/or as a prophylactic immunogenic composition. In various embodiments, the pharmaceutical compositions employ a self-adjuvanting immunogenic composition which contains an immunogen of the Formula I, II, IIIa and/or IIIb above, and a pharmaceutically acceptable carrier. Desirably, the immunogen prepared as described above is dissolved or suspended in an acceptable carrier, preferably an aqueous carrier.

As defined herein, the pharmaceutically acceptable carrier suitable for use in these immunogenic compositions are well known to those of skill in the art. In one embodiment, a preferred pharmaceutical carrier contains water for injection with mannitol added for tonicity at a concentration of about 45 mg/mL. Such carriers include, without limitation, water, buffered water, buffered saline, such as 0.8% saline, phosphate buffer, 0.3% glycine, hyaluronic acid, alcoholic/aqueous solutions, emulsions or suspensions. Other conventionally employed diluents, adjuvants and excipients, may be added in accordance with conventional techniques. Such carriers can include ethanol, polyols, and suitable mixtures thereof, vegetable oils, and injectable organic esters. Buffers and pH adjusting agents may also be employed. Buffers include, without limitation, salts prepared from an organic acid or base. Representative buffers include, without limitation, organic acid salts, such as salts of citric acid, e.g., citrates, ascorbic acid, gluconic acid, carbonic acid, tartaric acid, succinic acid, acetic acid, or phthalic acid, Tris, trimethanmine hydrochloride, or phosphate buffers. Parenteral carriers can include sodium chloride solution, Ringer's dextrose, dextrose and sodium chloride, laced Ringer's or fixed oils. Intravenous carriers can include fluid and nutrient replenishers, electrolyte replenishers, such as those based on Ringer's dextrose and the like. Preservatives and other additives such as, for example, antimicrobials, antioxidants, chelating agents, inert gases and the like may also be provided in the pharmaceutical carriers. These immunogenic compositions are not limited by the selection of the carrier. The preparation of these pharmaceutically acceptable compositions, from the above-described components, having appropriate pH isotonicity, stability and other conventional characteristics is within the skill of the art. See, e.g., texts such as Remington: The Science and Practice of Pharmacy, 20th ed, Lippincott Williams & Wilkins, publ., 2000; and The Handbook of Pharmaceutical Excipients, 4^{th} edit., eds. R. C. Rowe et al, APhA Publications, 2003.

Optionally, the pharmaceutical compositions can also contain a mild adjuvant, such as an aluminum salt, e.g., aluminum hydroxide or aluminum phosphate.

The concentration of immunogens of Formula I, II, IIIa and/or IIIb in the pharmaceutical formulations can vary widely, i.e., from less than about 0.1 mg/mL, usually at least 2 mg/mL, alternatively at least about 5 mg/mL to as much as 10 mg/mL, up to 20 mg/mL, and still alternatively, up to 50 mg/mL or more by weight, and will be selected primarily by fluid volumes, viscosities, etc., in accordance with the particular mode of administration selected.

A human unit dose form of the immunogenic composition is typically included in a pharmaceutical composition that comprises a human unit dose of an acceptable carrier, preferably an aqueous carrier, and is administered in a volume of fluid that is known by those of skill in the art to be used for administration of such compositions to humans (see, e.g., Remington's Pharmaceutical Sciences, 17th Edition, A. Gennaro, Editor, Mack Publishing Co., Easton, Pa., 1985).

These compositions may be sterilized by conventional, well known sterilization techniques, such as sterile filtration for biological substances. Resulting aqueous solutions may be packaged for use as is. In certain embodiments in which at least one polar sequence, e.g., -K-K-K-K-K-K- (SEQ ID NO: 36), is present in the immunogens of the composition, the aqueous solutions are lyophilized, the lyophilized preparation being combined with a sterile solution prior to administration.

Thus, as yet another aspect is a method of inducing in vivo the production of anti-Aβ15 or Aβ10 antibodies with a GMT of greater than 50,000 or greater than 300,000 or greater than 1,000,000, as provided above. In one embodiment, this method is accomplished without the use of any extrinsic adjuvant. In one embodiment, the pharmaceutical compositions may be therapeutically administered to a human subject diagnosed with AD. The pharmaceutical compositions are useful to reduce plaque formation and minimize progression to AD. In another embodiment, the pharmaceutical compositions are administered to healthy subjects as a prophylactic immunogenic composition for prevention of AD.

This method involves administering to a subject an effective antibody-inducing amount of the pharmaceutical compositions described herein, so as to induce anti-Aβ15 or Aβ10 antibody with a GMT of 50,000, greater than 100,000, greater than 500,000 or greater than 1,000,000. As described above, this method induces antibodies with much higher GMT as well. In patients already suffering from AD, this method can reduce progression of AD. The method can involve repeatedly administering the composition but at infrequent intervals, e.g., every 6 months. In subjects already suffering from AD, the induced anti-Aβ antibodies block assembly of the Aβ peptide into pathological forms and/or the deposition of Aβ peptide in plaques. Importantly, the neurotoxic effect of free Aβ is minimized by antibody binding. In healthy patients, the prophylactic immunogenic composition provides the immunized subject with high levels of antibodies which remove or reduce soluble Aβ in circulation and thereby prevents neurotoxicity and the deposition of plaques within the brain.

In one embodiment of this method, the route of administration of these pharmaceutical compositions is subcutaneous injection. Other suitable routes of administration include, but are not limited to, intramucosal, such as intranasal, oral, vaginal, or rectal, and parenteral, intradermal, transdermal, intramuscular, intraperitoneal, intravenous and intraarterial. The appropriate route is selected depending on a variety of considerations, including the nature of the composition, i.e., as a prophylactic immunogenic composition, and an evaluation of the age, weight, sex and general health of the patient and the components present in the immunogenic composition, and similar factors by an attending physician.

Similarly, suitable doses of the self-adjuvanting immunogenic compositions are readily determined by one of skill in the art, whether the patient is already infected and requires therapeutic treatment or prophylactic immunogenic composition treatment, the health, age and weight of the patient. The method and routes of administration and the presence of additional components in the compositions may also affect the dosages and amounts of the compositions. Such selection and upward or downward adjustment of the effective dose is within the skill of the art. The amount of composition required to produce a suitable response in the patient without significant adverse side effects varies depending upon these factors. Suitable doses are readily determined by persons skilled in the art. A suitable dose is formulated in a pharmaceutical composition, as described above (e.g., dissolved in about 0.1 mL to about 2 mL of a physiologically compatible carrier) and delivered by any suitable means. Dosages are typically expressed in a “unit dosage”, which is defined as dose per subject, e.g., a unit dosage of 1 mg immunogen. Alternatively dosages can be expressed as amount per body weight of the subject or patient, using the norm for therapeutic conversions as 80 kg body weight. For example, a 1 mg unit dose per subject is equivalent to about 12.5 μg/kg body weight.

In one embodiment, the intended therapeutic or prophylactic effect is conferred by a priming/boosting dosing regimen. For example, the dosage for an initial therapeutic administration or for a first priming therapeutic or prophylactic immunogenic composition administration in one embodiment is a “unit dosage” of less than about 0.1 mg to 100 mg of immunogen. In one embodiment, the unit dosage is 0.1 mg. In another embodiment, the unit dosage is 1 mg. In still another embodiment, the unit dosage is 10 mg. In still other embodiments, the unit dosage is as low as 0.01 mg. Thus, the initial priming dosage for a human, in certain embodiments, can range from very low unit dosages of at least about 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, to higher dosages of at least 1 mg, at least 3 mg, at least 5 mg, at least 7 mg, at least 10 mg, at least 12 mg, at least 15 mg, at least 20 mg. Still other human dosages range from between 21-30 mg, 31-40 mg, 41-50 mg/70-80 kg subject. Even higher dosages may be contemplated.

In one embodiment, the boosting dosages for either therapeutic prophylactic immunogenic composition or prophylactic immunogenic composition use are the same as the above described priming dosage. The same specific unit dosage or unit dosage ranges as for the priming dosage above may be employed for the boosting dosage. Thus, the boosting dosage for a human, in certain embodiments, can occur in a unit dosage range a “unit dosage” of less than about 0.1 mg to 100 mg of immunogen. In one embodiment, the unit dosage is 0.1 mg. In another embodiment, the unit dosage is 1 mg. In still another embodiment, the unit dosage is 10 mg. Thus, the booster unit dosage for a human, in certain embodiments, can range from very low unit dosages of at least about 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, to higher dosages of at least 1 mg, at least 3 mg, at least 5 mg, at least 7 mg, at least 10 mg, at least 12 mg, at least 15 mg, at least 20 mg. Still other human dosages range from between 21-30 mg, 31-40 mg, and 41-50 mg/70-80 kg subject. Even higher dosages may be contemplated.

In alternative embodiments, the boosting dosages are lower than the priming dosage identified above.

In one embodiment, the first “boosting” is administered within weeks of the initial priming dose. In one embodiment, the boosting dose is administered at least 3 weeks after the priming dose, followed by a re-boost administered not earlier than 3 weeks from the preceding boosting dose. In another embodiment, the first boosting dose is administered about 3 to 4 weeks following the priming dose. Additional boosting dosages are administered thereafter at least 3 weeks thereafter, more suitably about 6 months to one or more years, following the first booster dose. In another embodiment of an administration protocol, a priming dosage of a self-adjuvanting immunogenic composition as described herein is administered which is about 10 mg. The subsequent first boosting dosage (e.g., 0.01-10 mg) is then administered at least three weeks after the priming dosage. Thereafter, additional boosting dosages are administered every 6 months to one year from the preceding boosting dosage.

The timing and dosage of any priming/boosting regimen may be selected by the attending physician depending upon the patient's response and condition as determined by measuring the specific anti-Aβ antibody titer obtained from the patient's blood, as well as normal considerations related to the physical condition of the patient, e.g., height, weight, age, general physical health, other medications, etc.

In one embodiment of the prophylactic/therapeutic method involves administering a priming effective amount of the immunogenic composition in a unit dosage of less than or about 10 mg, and following up the administration by two boosters administered at weeks 3 and weeks 6 at the same effective unit dosage. This method induces antibodies of a GMT greater than 100,000, optionally without any extrinsic adjuvant.

In another embodiment of the prophylactic/therapeutic method involves administering a priming effective amount of the immunogenic composition in a unit dosage of less than or about 10 mg, and following up the administration by two boosters administered at weeks 3 and weeks 6 at the same effective dosage. This method induces antibodies of a GMT greater than 1,000,000 optionally without any extrinsic adjuvant.

Administration desirably continues until at least clinical symptoms or laboratory tests indicate that disease has stopped progressing and for a period thereafter. The dosages, routes of administration, and dose schedules are adjusted in accordance with methodologies known in the art.

For use as a prophylactic immunogenic composition, the priming and boosting dosages are similar to the boosting dosages of the therapeutic immunogenic composition, but are administered at certain defined intervals from about three weeks to six months after the initial administration of the composition. Possibly additional administrations may be desirable thereafter.

As indicated in the examples below, the antibody with high GMT induced by the exemplary pharmaceutical or immunogenic compositions described herein may reduce the need for a high frequency of boosting dosages for either therapeutic or vaccinal use.

In still another embodiment of the methods described herein, the compositions may be used in conjunction with, or sequentially with, other therapies or pharmaceutical regimens to treat AD.

The following examples illustrate certain embodiments of the above-discussed compositions and methods. These examples do not limit the disclosure of the claims and specification.

### IV. Examples

### Example 1

### Generation of an Immunogenic Composition of the Invention

A. Experimental Immunogens

Various immunogenic compositions as described above were prepared containing a single Aβ15 peptide component, a T cell helper sequence, linker amino acids (italicized only) and the Pam2C- and Pam3C-lipoprotein cap according to the formula of SEQ ID NO: 25. The NAc(Pam2C) capped formula is to be prepared similarly to the Pam2C and Pam3C capped immunogens described by this formula:

SEQ ID NO: 25:
(Pam2C or Pam3C or NAc(Pam2C))-S-S-Q-Y-I-K-A-N-S-
K-F-I-G-I-T-E-L-D-A-E-F-R-H-D-S-G-Y-E-V-H-H-Q-
amide.

Alternative immunogens are prepared in a similar manner for other similar compositions which are expected to produce similar results. Such similar immunogens include, e.g.,:

SEQ ID NO: 26:
Pam2C-S-S-K(Q-Y-I-K-A-N-S-K-F-I-G-I-T-E-L-)-S-D-A-
E-F-R-H-D-S-G-Y-E-V-H-H-Q-amide
or
SEQ ID NO: 27:
Pam2C-S-S-K(D-A-E-F-R-H-D-S-G-Y-E-V-H-H-Q)-S-Q-Y-
I-K-A-N-S-K-F-I-G-I-T-E-L-amide.

The Pam2C- and Pam3C immunogens prepared according to the first of the three preceding formulae were synthesized by Bachem Biosciences, Inc. or Mimotopes, Pty. Ltd. or Anaspec, Inc. using conventional solid phase synthesis techniques and automated synthesizers. Commencing with the appropriate amide resin, e.g., an amidated C-terminal Gln as the C-terminus the cycles of synthesis proceeded towards the N-terminus of the Aβ peptide, through the linker amino acids, through the helper T cell epitope sequence (which preferably is the tetanus toxoid promiscuous T helper sequence Q-Y-I-K-A-N-S-K-F-I-G-I-T-E-L SEQ ID NO: 12) and through Ser-Ser (or optional polar sequence) at the N-terminus for the first immunogen above.

Tripalmitoyl-S-glyceryl-cysteine, and fmoc protected di-palmitoyl-S-glyceryl-cysteine were synthesized by Bachem and were coupled to the N-terminal serine of the nascent peptide chain as indicated. Other synthetic methods known in the art may also be employed. Trifluoroacetic acid was used to cleave the lipopeptide from the resin and deprotect the peptide. The resulting immunogenic product was dried, then taken into aqueous solution, converted to an acetate salt form and dried.

The second and third immunogens above were prepared in a similar manner, depending upon which amino acid sequence was coupled to the resin.

The final products were checked by amino acid analysis for the appropriate content of amino acids, and by mass spectroscopy. Purity was estimated in general to be around 70%, but the resulting lipopeptides were not purified further in the examples provided. The experimental immunogens synthesized and studied are

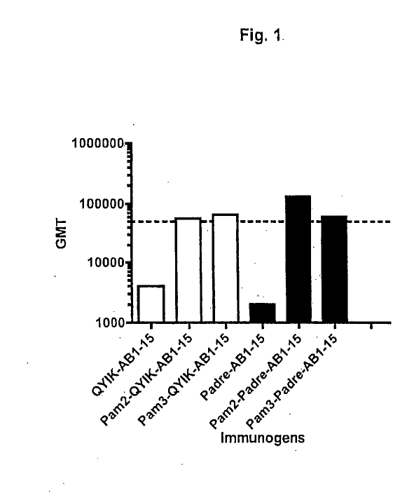

SEQ ID NO: 19:
Pam2C-S-S-Q-Y-I-K-A-N-S-K-F-I-G-I-T-E-L-D-A-E-F-R-
H-D-S-G-Y-E-V-H-H-Q-amide (identified as Pam2-
QYIK-Aβ15 in FIG. 1);
SEQ ID NO: 20:
Pam3C-S-S-Q-Y-I-K-A-N-S-K-F-I-G-I-T-E-L-D-A-E-F-R-
H-D-S-G-Y-E-V-H-H-Q-amide (identified as Pam3-
QYIK-Aβ15 in the FIG. 1);
SEQ ID NO: 21:
Pam2C-S-S-Xaa1-K-Xaa2-V-A-A-W-T-L-K-A-A-Xaa3-D-A-
E-F-R-H-D-S-G-Y-E-V-H-H-Q-amide, wherein Xaa1 and
Xaa3 are D-Alanine and Xaa2 is L-cyclohexylalanine
(identified as Pam2-Padre-Aβ15 in FIG. 1);
and
SEQ ID NO: 22:
Pam3C-S-S-Xaa1-K-Xaa2-V-A-A-W-T-L-K-A-A-Xaa3-D-A-
E-F-R-H-D-S-G-Y-E-V-H-H-Q-amide, wherein Xaa1 and
Xaa3 are D-Alanine and Xaa2 is L-cyclohexylalanine
(identified as Pam3-Padre-Aβ15 in the FIG. 1).

B. Control Immunogens

The synthesis procedures described above were also employed to prepare other immunogens for comparison with the experimental immunogens discussed above for use in the following examples. Among such “control” immunogens were two immunogens that contained only a T cell helper sequence fused to the Aβ15, i.e. a formula of

SEQ ID NO: 23:
Q-Y-I-K-A-N-S-K-F-I-G-I-T-E-L-S-D-A-E-F-R-H-D-S-G-
Y-E-V-H-H-Q-amide (identified as QYIK-Aβ1 in FIG.
1)
or
SEQ ID NO: 24:
Xaa1-K-Xaa2-V-A-A-W-T-L-K-A-A-Xaa3-D-A-E-F-R-H-D-
S-G-Y-E-V-H-H-Q-amide, wherein Xaa1 and Xaa3 are
D-Alanine and Xaa2 is L-cyclohexylalanine
(identified as Padre-Aβ15 in FIG. 1).

### Example 2

### Immunization Protocol

The following immunization protocol was used for the immunogens of Example 1. Animals were purchased from Harlan Laboratories and acclimated at Molecular Diagnostic Services, Inc. for at least 1 week before immunization. Both BALBc and C57BL6/BALBc F1 mice were used. Immunogens prepared as described in Example 1 (using Pam2CSS and Pam3CSS) were taken up in dimethylsulfoxide (DMSO), then diluted to 10% DMSO in phosphate buffered saline. This provided opalescent, turbid solutions with no macroscopic particulate. Unless otherwise stated mice were immunized IP with 1 mg of Immunogen at Day 0 (Prime) and the same at Week 2 (Boost) and serums were obtained at Week 4 for titration. GMT of the animal sera used in the examples below was greater than 50,000.

Note that the tet toxoid T cell helper sequence used in the immunogens discussed herein was originally discovered as having promiscuous helper activity in human cells and appears to have wide activity in multiple animal species.

Similar results are expected when the lipopeptide cap of the immunogens is aceylated Pam2CSS.

### Example 3

### Serum Titrations

After immunizations with the immunogens of Example 1, serums were assayed to determine anti-Aβ peptide titers by conventional ELISA methods. Briefly, Maxisorp Immuno plates, coated with streptavidin, were coated with 2 ng/well Aβ15 and incubated at 22° C. for 1 hour or 4° C. overnight. After thorough washing with 0.1% triton X 100 buffer, blocking with 1% bovine serum albumin (BSA) and rewashing, serum dilutions were applied and incubated at 22° C. for 1 hour. After incubation, the plates were thoroughly washed with triton-X 100 buffer again and 1/10,000 goat anti-mouse IgG-horseradish peroxidase (HRP) conjugate (or the appropriate reagent for rat or rabbit) was applied. The plates were then incubated for 1 hour at 22° C. then washed thoroughly and developed with ABTS for 45 minutes at room temperature on a shaker table. Absorbance was measured at 405 nm. Control wells containing 1/60000 normal mouse serum were measured; and the reciprocal of the lowest dilution of test serums with an OD greater than the mean+8 SDs of controls was taken as the titer.

A. Comparison of Geometric Mean Antibody Titers (GMT) in Animals Immunized with the Experimental Immunogens vs. with Control Immunogens.

FIG. 1 is a graph showing the geometric mean titers (GMT) of immune serums from immunized mice. The immunogens identified under the X axis of FIG. 1 were synthesized as described in Example 1 and are identified in that example. The immunogens used as the T helper sequence either the tetanus toxoid promiscuous T helper sequence described for use in man (Q-Y-I-K-A-N-S-K-F-I-G-I-T-E-L SEQ ID NO: 12) or the PADRE T helper sequence engineered for promiscuous human DR binding (Xaa1-K-Xaa2-V-A-A-W-T-L-K-A-A-Xaa3 SEQ ID NO:28, wherein Xaa1 and Xaa3 were each D-Alanine, and Xaa2 was L-cyclohexylalanine.

The results of FIG. 1 show that the immune serums reacted similarly with the four experimental lipopeptide immunogens, demonstrating serums with antibodies with GMTs of 50,000 or greater. In contrast, immunogens lacking the Pam2CSS- or Pam3CSS-N-terminal cap induced antibodies with maximal GMTs of 4-5,000.

B. Effect of Booster Dose of Experimental Immunogen on GMT

In an experiment to determine the effect of booster dosages, three mice were immunized with experimental immunogen, Pam2C-S-S-Q-Y-I-K-A-N-S-K-F-I-G-I-T-E-L-D-A-E-F-R-H-D-S-G-Y-E-V-H-H-Q-amide (SEQ ID NO: 13) in a protocol of a 1 mg intraperitoneal (IP) priming dose at Day 0, followed by a 1 mg IP booster at Week 3, and a 1 mg IP second booster at Week 6. Mice were bled at weeks 5 and 8. The serums are titered on synthetic Aβ40. The GMT at 5 weeks was 3 million and the GMT at 8 weeks was 8 million. When these serums were used for depletion experiments, discordance between titers and depletions obtained suggested that these titer results may have been incorrectly high. This data is being reevaluated.

The anti-Aβ antibody titers are expected to decline somewhat after week 8 and remain steady at a high titer through about week 28. The titers are expected to show a marked elevation about 2 weeks a delayed (e.g., 6 month) boost.

### Example 4

### Inhibition of Free β-Amyloid 40 Concentration by Antibody

Antibody treatment of toxins is predicated on the binding of the toxin by high affinity antibody lowering the concentration of free toxin and thus blocking the toxicities associated with the toxin (Nowalowski et al. 2002 Proc Natl Acad Sci USA 99:11346). This holds true both in vitro and in vivo (ibid). Accordingly, an assay to measure the lowering of free Aβ40 concentrations by immune anti-Aβ15 or Aβ10 serum was performed, based on the premise that this endogenously produced protein was acting as a toxin.

The principle of this Aβ40 ELISA assay is, firstly, to bind the anti-A 15 immune serum, in parallel with normal mouse serum (NMS) controls, onto protein A coated beads (Pierce ImmunoPure Protein A plus resin). This was done at the required dilutions and the tubes were incubated on a tube rotator for at least 1 hour at RT. The tubes were then spun at 6,000 rpm in a microfuge for 30 seconds to pellet the beads and remove the serum sample. The beads were then washed thoroughly. The Aβ40 solution (200 ng/ml) was added. Tubes were incubated on the tube rotator for at least 1 hour at RT. The tubes were then centrifuged at 6,000 rpm for 30 seconds to pellet the resin and the supernatant was removed to a fresh tube for analysis.

The Aβ40 ELISA (Invitrogen) was used according to the manufacturer's instructions, except that the Aβ40 used in the assay was also used to generate the standard curve. The unbound Aβ40 concentration in the test and control supernatants was then used to calculate the % inhibition of free Aβ40 concentration in the anti-Aβ15 or Aβ10 exposed conditions by comparison with the control concentration.

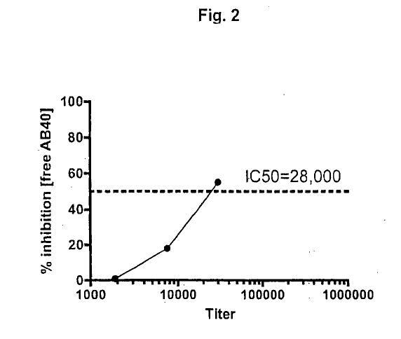

FIG. 2 shows the dose response curve of inhibition of Aβ40 concentration in relation to antibody titer for the immunogens of Example 1. These data show 50% inhibition of Aβ40 concentration at an antibody titer of 28,000, a titer below the GMT obtained with the 1 mg prime/boost regimen (no adjuvant) used with the effective immunogens. For technical reasons, the serum needed to be diluted at least 10-fold to perform this depletion assay. However, it can be seen from the trend of the curve that the undiluted serum titer would provide considerably more inhibition of free amyloid β levels.

The antibody titers achieved compare favorably with the antibody titers obtained in humans (2,200-4,000) in a phase 1/2a study that showed evidence of efficacy (Gilman et al. 2005 Neurology 64:1553) and also in mouse studies (5,000-50,000, using CFA/IFA) showing efficacy in animal models (Bard et al. 2003 Proc Natl Acad Sci USA 100:2023).

These findings provide evidence that the presently attained titers produce an even greater reduction in Aβ40 concentration than that attained in previously reported human and animal model studied. These data further suggest that even greater improvement in neurological parameters may be expected.

It is noteworthy that a number of publications (Solomon et al., 1997 Proc Natl Acad Sci USA 94:4109; Frenkel et al., 1998 J Neuroimmunol 88:85; McLaurin et al, 2002 Nat Med 8:1263; Bard et al., 2003 Proc Natl Acad Sci USA 100:2023; Levites et al., 2006 J Clin Invest 116:193) emphasize that antibody binding to soluble Aβ40 alone does not ensure activity in assays measuring binding to aggregated amyloid in plaques, prevention of amyloid aggregation, solubilization of aggregated amyloid, and prevention of neurotoxicity. Instead, binding to epitopes within the N-terminal 11 amino acids of Aβ is associated with these activities, i.e., precisely the antibodies being induced exclusively by immunization with Aβ15 or Aβ10 in the present immunogens.

### Example 5

### Generation of an Immunogenic Composition of the Invention with Polar Sequences

Immunogens are synthesized using similar methodologies described in Example 1, except that the immunogens contain a polar charged sequence of four lysine residues inserted between the serines at the carboxy terminal end of the lipopeptide, i.e.,

SEQ ID NO: 30

Pam2C-S-K-K-K-K-S-Q-Y-I-K-A-N-S-K-F-I-G-I-T-E-L-D-
A-E-F-R-H-D-S-G-Y-E-V-H-H-Q-amide
or

(SEQ ID NO: 34)

Pam2C-S-K-K-K-K-S-Q-Y-I-K-A-N-S-K-F-I-G-I-T-E-L-D-
A-E-F-R-H-D-S-G-Y amide.

### Example 6

### Titers—Effects of Vaccine Construct, Dose, Frequency and Species

Rats (n=3) were immunized by subcutaneous injection with 10 mg/rat with the Pam2C-S-K-K-K-K-S-Q-Y-I-K-A-N-S-K-F-I-G-I-T-E-L-D-A-E-F-R-H-D-S-G-Y-E-V-H-H-Q amide (SEQ ID NO: 30) immunogen described in the Example 5 above. The initial priming dose was administered at day 0. The rats were bled at week 2. The geometric mean titers (3 rats) against free amyloid β 1-40 was 60,000.

Thereafter two booster administrations of 10 mg/rat are administered at week 3 and at week 6. The rats are bled, and titers are determined two weeks after each boost, i.e., at week 5 and week 8. The geometric mean titers (3 rats) against amyloid β 1-40 are anticipated to be greater than about 500,000 at 5 weeks and greater than 2,000,000 at 8 weeks after the first injection. The three weekly spacing is expected to greatly enhance the antibody response.

In a second experiment, rats (n=3) were immunized by subcutaneous injection with 1 mg/rat with the Pam2C-S-K-K-K-K-S-Q-Y-I-K-A-N-S-K-F-I-G-I-T-E-L-D-A-E-F-R-H-D-S-G-Y-E-V-H-H-Q-amide (SEQ ID NO: 30) immunogen described in the Example 5 above. The initial priming dose was administered at day 0. The rats were bled at week 2. The geometric mean titer (3 rats) against free amyloid β 1-40 was 19,000.

Thereafter two booster administrations of 1 mg/rat are administered at week 3 and at week 6. The rats are bled, and titers are determined two weeks after each boost, i.e., at week 5 and week 8. The geometric mean titers (3 rats) against amyloid β 1-40 are anticipated to be greater than about 50,000 at 5 weeks and greater than 100,000 or greater than 500,000 at 8 weeks after the first injection. The three weekly spacing is expected to greatly enhance the antibody response.

### Example 7

### Insertion of Polar Spacer Group Enhances Aqueous Solubility

The immunogens of Example 1 (with either Pam3CSS- or Pam2CSS-lipopeptide caps) were poorly soluble in aqueous solvents. They were formulated for administration by dissolving them in dimethylsulphoxide (DMSO) and diluting with phosphate buffered saline to 5-10% DMSO. This yielded an opalescent somewhat turbid solution that was injected into animals.

The immunogen of Example 5 containing the polar, charged -K-K-K-K- (SEQ ID NO: 35) sequences at the carboxy terminus of the capped lipopeptide(s) and the B cell epitope Aβ15 was synthesized and had a much improved solubility, yielding clear solutions in water for injection (WFI). Addition of mannitol to establish isotonicity (see the “solutions” in the Table 1 below) in water for injection did not impair solubility. Brief incubation at 37° C. was required to solubilize the lipopeptide, but a clear solution remained after overnight storage at 4° C.

The solubility of several solutions of the immunogen containing the polar charged spacer are reported in Table 1 below.

TABLE 1
ENHANCED SOLUBILITY OF PAM2CSKKKKS-
(SEQ ID NO: 44) CAPPED IMMUNOGEN

| Solution | pH | Appearance | Comments |
|---|---|---|---|
| 2 mg/mL-30 mg/mL | 5 | Clear colorless | Soluble with 37° C. |
| mannitol | | | incubation |
| 10 mg/mL-30 mg/mL | 5 | Clear slight straw | Soluble with 37° C. |
| mannitol | | color | incubation |
| 30 mg/mL-30 mg/mL | 5 | Clear slight straw | Soluble with 37° C. |
| mannitol | | color | incubation |
| 2 mg/mL WFI | 5 | Clear colorless | Soluble with 37° C. |
| | | | incubation |
| 10 mg/mL WFI | 5 | Clear colorless | Soluble with 37° C. |
| | | | incubation |
| 30 mg/mL WFI | 5 | Clear slight straw | Soluble with 37° C. |
| | | color | incubation |

### Example 8

### GMT for Rats Immunized with Immunogen Containing Charged, Polar Sequence

Three rats per group are immunized with a different dosage per group of the experimental immunogens:

SEQ ID NO: 30

Pam2C-S-K-K-K-K-S-Q-Y-I-K-A-N-S-K-F-I-G-I-T-E-L-D-
A-E-F-R-H-D-S-G-Y-E-V-H-H-Q-amide
or

(SEQ ID NO: 34)

Pam2C-S-K-K-K-K-S-Q-Y-I-K-A-N-S-K-F-I-G-I-T-E-L-D-
A-E-F-R-H-D-S-G-Y-amide.

These immunogens are referred to as “TABI15-K4” or TABI10-K46”, respectively. Each rat is administered a dose of either 0.1 mg TABI15-K4 or TABI10-K4, or 1 mg TABI15-K4 or TABI10-K4 or 10 mg TABI15-K4 or TABI10-K4 at day 0. Each rat is boosted with the same dose at week 3 and then again at week 6. The serum antibody titers for each rat are measured at weeks 5 and 8 against the full length recombinant amyloid β protein.

The results are anticipated to show that by week 5, all of the doses provide GMTs of about 500,000 or about 1,000,000 (for higher mg doses). All of these titers are associated with greater than 99% reduction of free amyloid β protein levels. A more rapid initial titer ascent is anticipated in the 10 mg group, but the rats administered 1 mg/rat are anticipated to achieve the same titer over 5 weeks. It is anticipated that titers taken after a second boost to be administered at 6 weeks will exceed 1,000,000.

Based upon this data, it is anticipated that these immunogens will permit a ten to 100-fold dose reduction in the vaccine amount required to achieve a successful therapeutic titer. For example, doses at low at 0.1 mg are likely to achieve therapeutically effective titers. Such low therapeutically effective doses will provide dramatic and unexpected therapeutic and cost benefits to the patient population worldwide.

Numerous modifications and variations of the embodiments illustrated above are included in this specification and are expected to be obvious to one of skill in the art. Such modifications and alterations to the compositions and processes described herein are believed to be encompassed in the scope of the claims appended hereto. All documents listed or referred to above, as well as the attached Sequence Listing, are incorporated herein by reference.

What is claimed is:

1. A self-adjuvanting immunogenic composition comprising an immunogen comprising:
(a) an amyloid β B cell epitope located within the amino acid sequence of amino acids 1-17 of SEQ ID NO: 1;
(b) a universal T helper sequence (R1); and
(c) a lipopeptide cap (R2) selected from the group consisting of a dipalmitoyl-S-glyceryl-cysteine, a N-acetyl (dipalmitoyl-S-glyceryl cysteine), and a tripalmitoyl-S-glyceryl cysteine.

2. The composition according to claim 1, wherein said B cell epitope has a sequence selected from the group consisting of
-D-A-E-F-R-H-D-S-G-Y-E-V-H-H-Q-, amino acids 1-15 of SEQ ID NO: 1 and D-A-E-F-R-H-D-S-G-Y, amino acids 1-10 of SEQ ID NO: 1.

3. The composition according to claim 1 having a formula of R2-(R1-Aβ B cell epitope).

4. The composition according to claim 1 having a formula of
R2-(Aβ B cell epitope-R1) or R2-K(Aβ B cell epitope)-R1 or R2-K(R1)-Aβ B cell epitope.

5. The composition according to claim 1 further comprising a linker sequence of from one to ten amino acids wherein said linker sequence is attached to the Cys of R2 to link R2 to other components or is located between other components of said immunogen.

6. The composition according to claim 3, wherein R2 is linked to an α-amino function at the amino terminus of R1.

7. The composition according to claim 3, wherein R2 is linked to an ε-amino of a K residue located between R1 and the amino terminus of said Aβ B cell epitope.

8. The composition according to claim 3, wherein R2 is linked to an ε-amino of a K residue located at the C terminus of said Aβ B cell epitope.

9. The composition according to claim 1, wherein said immunogen further comprises a sequence of charged polar amino acids optionally flanked by one or more neutral linker amino acids.

10. The composition according to claim 9, wherein said charged polar amino acid sequence is located at a position selected from the group consisting of (a) as part of the carboxy terminus of R2; (b) between R1 and said B cell epitope; (c) at the free carboxy terminus of R1 or said B cell epitope; (d) at the free amino terminus of said R1 or said B cell epitope; (e) before or after the inserted K.

11. The composition according to claim 5, wherein said linker is selected from the group consisting of -S-, -S-S-, -G-, and -G-S-.

12. The composition according to claim 9, wherein said polar sequence comprises a sequence of from 4 to 8 amino acids selected individually from the group consisting of K, E, D, and R, which is optionally flanked by said linker amino acids.

13. The composition according to claim 12, wherein said polar sequence is selected from the group consisting of -K-K-K-K- (SEQ ID NO:35), -S-K-K-K-K-S- (SEQ ID NO: 39), G-K-K-K-K-G (SEQ ID NO: 41),
-S-K-K-K-K-K-K-S (SEQ ID NO: 40), and G-K-K-K-K-K-K-G (SEQ ID NO: 42).

14. The composition according to claim wherein said R1 sequence is selected from the group consisting of:
(a) Q-Y-I-K-A-N-S-K-F-I-G-I-T-E-Xaa SEQ ID NO: 3, wherein said Xaa is absent or L, with an optional amino acid linker;
(b) Xaa1-K-Xaa2-V-A-A-W-T-L-K-A-A-Xaa3 SEQ ID NO: 28,
wherein Xaa1 and Xaa3 are each a D-Alanine and Xaa2 is L-cyclohexylalanine; and
(c) F-N-N-F-T-V-S-F-W-L-R-V-P-K-V-S-A-S-H-L-E- SEQ ID NO: 4.

15. The composition according to claim 1, wherein, in each immunogen, R2 is selected from the group consisting of dipalmitoyl-S-glyceryl-cysteine or N-acetyl (dipalmitoyl-S-glyceryl cysteine) or tripalmitoyl-S-glyceryl cysteine; R2 further comprises an optional amino acid linker sequence of -S-S- residues; and R1 is Q-Y-I-K-A-N-S-K-F-I-G-I-T-E-L SEQ ID NO: 47 with an optional amino acid linker of -S-linking it to the B cell epitope.

16. The composition according to claim 1, wherein, in each immunogen, R2 is dipalmitoyl-S-glyceryl-cysteine or N-acetyl (dipalmitoyl-S-glyceryl cysteine) or tripalmitoyl-S-glyceryl cysteine, and an amino acid linker sequence of -S-S- residues; and R1 is Xaa1-K-Xaa2-V-A-A-W-T-L-K-A-A-Xaa3 SEQ ID NO: 28, wherein Xaa1 and Xaa3 are each a D-Alanine and Xaa2 is L-cyclohexylalanine, with an optional amino acid linker of -S-.

17. A pharmaceutical composition comprising the self-adjuvanting immunogenic composition of claim 1, and a suitable pharmaceutical carrier or excipient, wherein said composition induces in an immunized subject anti-Aβ antibodies with a geometric mean titer of greater than 50,000.

18. The pharmaceutical composition according to claim 23, wherein said geometric mean titer is greater than 1,000,000.

19. A method of inducing in vivo the production of anti-Aβ peptide antibodies comprising immunizing a subject with an effective antibody-inducing amount of the composition of claim 17, wherein said effective amount induces anti-Aβ antibodies with a geometric mean titer of 50,000 or greater.

20. The method according to claim 19, comprising administering to said subject an initial priming effective amount of said composition, followed by a booster effective amount of said composition.

21. The method according claim 19, wherein said effective amount is 10 mg or less.

22. The method according to claim 19, wherein said effective amount is 0.1 mg or less.
